(19) United States
(12) Patent Application Publication
(10) Pub. No.: US 2020/0087258 A1
(43) Pub. Date: Mar. 19, 2020

(54) INHIBITOR OF CITRATE TRANSPORTER AND THEIR USE IN THERAPY

(71) Applicant: Eternygen GmbH, Berlin (DE)

(72) Inventors: Grit Zahn, Berlin (DE); Steve Bromidge, Goring on Thames (GB); Chris Yarnold, Oxfordshire (GB); Sabine Schaertl, Hamberg (DE); Someina Khor, Reading (GB)

(21) Appl. No.: 16/495,582

(22) PCT Filed: Mar. 19, 2018
(86) PCT No.: PCT/EP2018/056827

§ 371 Date: Sep. 19, 2019

(30) Foreign Application Priority Data
Mar. 20, 2017 (GB) 1704357.1

(51) Int. Cl. C07D 211/96; C07D 401/12; C07D 241/04

(52) U.S. Cl. CPC C07D 211/96; C07D 241/04; C07D 401/12

(57) ABSTRACT

The present invention relates to a compound of general formula (I) for use in treating diseases depending on the activity of a citrate transporter, wherein the compound has the general formula (I)

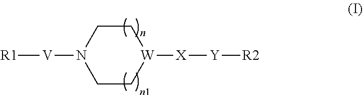

wherein R1 is Aryl, substituted aryl, styryl, or bicyclic; V is SO₂; W is N with n and n1 are 1 and X is C═O, or W is C with n and n1 are 0 or 1 and X is CH₂ or C═O, Y is N—H or N-methyl and R2 is benzyl, substituted benzyl or CH₂(2-pyridyl). Further, it relates to the use of a compound of general formula (I) for preparing a medicament and its use for the treatment of obesity and diabetes, in particular type 2 diabetes and other metabolic diseases as well as for the treatment of age related diseases.

### FIELD OF THE INVENTION

The present invention relates to the treatment of diseases depending on the activity of citrate transporters, its use for preparing a medicament and its use for the treatment of obesity and diabetes, in particular type 2 diabetes and other metabolic diseases as well as for the treatment of age-related diseases.

### BACKGROUND OF THE INVENTION

Energy balance and insulin action are both closely related to life span. Caloric excess leads to obesity and insulin resistance to an increased mortality. Caloric restriction reduces adiposity and increases lipid oxidation, insulin sensitivity, and mitochondrial biogenesis. In addition, caloric restriction reverses obesity, type 2 diabetes, delays aging, and prolongs life in many species, including primates (Hursting et al, 2003, Annu. Rev. Med. 54, p. 131-152; Lopez-Lluch et al, 2006, Proc. Natl. Acad. Sci. U.S.A 103, p. 1768-1773; Hunt et al, 2006, Ageing Res. Rev. 5, p. 125-143; Fontana and Klein 2007, JAMA 297, p 986-994; Colman et al, 2009, Science 325, p. 201-204).

Beneficial effects of caloric restriction are mediated by decreased plasma concentrations of anabolic hormones and growth factors, i.e. insulin and insulin like growth factors (Fontana and Klein 2007, JAMA 297, p 986-994; Colman et al, 2009, Science 325, p. 201-204). Reduced expression of the Indy (for I am Not Dead, Yet) gene in D. melanogaster and C. elegans has been shown to promote longevity in a manner akin to caloric restriction, however the cellular mechanism by which reduced expression of Indy leads to increased survival is unknown (Rogina et al, 2000, Science 290, p. 2137-2140; Fei et al, 2004, Biochem. J. 379, p. 191-198; Fei et al, 2003, J. Biol. Chem. 278, p. 6136-6144; Wang et al, 2009, Proc. Natl. Acad. Sci. U.S.A 106, p. 9262-9267).

Indy encodes in D. melanogaster a non-electrogenic dicarboxylate and citrate transporter (Knauf et al, 2006, Biochem. J. 397, p. 25-29; Knauf et al, 2002, Proc. Natl. Acad. Sci. U.S.A 99, p. 14315-14319) and it is mainly expressed in the fat body, mid gut, and oenocyte (Rogina et al, 2000, Science 290, p. 2137-2140), the major organs of intermediary metabolism in flies. In mammals, the gene product of SLC13A5, the sodium-coupled citrate transporter NaCT (mINDY), shares the highest sequence and functional similarity with INDY of D. melanogaster (Inoue et al, 2002, Biochem. J. 367, 313-319, WO 2004/048925) and it is predominantly expressed in liver cells (Inoue et al, 2002, J. Biol. Chem. 277, p. 39469-39476; Knauf et al, 2006, Biochem. J. 397, p. 25-29; Knauf et al, 2002, Proc. Natl. Acad. Sci. U. S. A 99, p. 14315-14319; Gopal et al, 2007, Am. J. Physiol Gastrointest. Liver Physiol 292, G402-G408, WO 2004/048925).

Indy and its mammalian homolog mINDY (Slc13a5, NaCT) are transporters of tricarboxylic acid (TCA) cycle intermediates. Basically, INDY handles the uptake of citrate via the plasma membrane into the cytosol where citrate is used for the synthesis of fatty acids and cholesterol (Inoue et al, 2002, J. Biol. Chem. 277, p. 39469-39476, Birkenfeld et al, 2011, Cell Metab 14, p. 184-195). Cytosolic citrate is known as the prime carbon source for the synthesis of fatty acids, triacylglycerols, cholesterols and low-density lipoproteins (Willmes and Birkenfeld 2013 Comput Struct Biotechnol J. 2013 6:7). Moreover citrate leads to the activation of fatty acid synthesis and affects glycolysis and β-oxidation (Spencer and Lowenstein 1962 J Biol Chem 237: 3640-48, Bloch and Vance 1977 Ann Rev Biochem 46:263-298, Ruderman et al. 1999 Am J Physiol 276: E1-18). Main organs for fatty acid synthesis are the liver and white adipose tissue and fatty acid synthesis has been shown to directly correlate with cytosolic citrate concentrations, partially depending on the direct import across the plasma membrane by mINDY (Inoue 2002 Biochem Biophys Res Comm 299:465-471, Gopal et al, 2007, Am. J. Physiol Gastrointest. Liver Physiol 292, G402-G408).

Birkenfeld and colleagues described in 2011 that deletion of the mouse homologue of INDY (mIndy) reduces citrate uptake into the liver and sterol and fatty acid synthesis in hepatocytes. Furthermore, it reduces adiposity, prevents lipid accumulation into liver and skeletal muscle and increases insulin sensitivity under high fat diet (HFD) conditions and during aging in mIndy knock-out mice. Loss of mINDY augments energy expenditure associated with increased hepatic fat oxidation and attenuates hepatic lipogenesis (Birkenfeld et al, 2011, Cell Metab 14, p. 184-195). Furthermore, Pesta and colleagues (Pesta et al. 2015 Aging 7(12), p. 1086-93) showed that a hepatic knockdown of mINDY in rats under HFD improved metabolism by reducing fasting plasma insulin, hepatic glucose production, liver fat accumulation and improving insulin sensitivity. Additionally, Rong et al. showed recently (Rong et al. 2015 Conference abstract Keystone Symposia: Obesity and the Metabolic Syndrome/Liver Metabolism March 2015) that hepatic knockdown of mINDY in mice improved several metabolic parameters such as fed glucose and insulin sensitivity assessed by HOMA-IR, reduced the body weight in animals under high fat diet mainly in liver and adipose tissue weights and reduced liver triglycerides.

Expression analysis of INDY in monkey and human liver samples showed that INDY expression is increased in human obesity and fatty liver as well as in monkeys after 2 years high fat diet (Loeffelholz et al. 2013 ADA poster 1868-P)

Reducing INDY expression by knockout and knockdown has been proven beneficial in terms of metabolic regulation and/or life span in all species tested so far. Therefore, mINDY is a drug target for the treatment of metabolic disease, such as obesity, non-alcoholic fatty liver disease (NAFLD), non-alcoholic steatohepatis (NASH) and type 2 diabetes, but also hyperlipidemia and hypercholesterolemia (Birkenfeld et al, 2011, Cell Metab 14, p. 184-195, Pesta et al. 2015 Aging 7(12), p. 1086-93, Mancusso et al, 2012, Nature 491, p. 622-626; Frankel and Rogina, 2012, Front Genet 3, p. 13; Schindler, 2012, Ther Adv Endocrinol Metab 3, p. 51-53, WO 2004/048925, Neuschafer-Rube Diabetes. 2014 63(3), p. 1048-57, Willmes 2016 Aging 2, p. 208-9). It can be expected that an inhibitor of mINDY function reducing the uptake of extracellular citrate will have similar beneficial therapeutic effect as reduction of INDY expression by knockout and knockdown mINDY (WO 2004/048925).

In fact, a very recent work from Huard et al. 2015 (Huard et al. 2015 Sci Rep. 5, p. 17391) has proven this hypothesis with a small molecule citrate analogue and have shown an improvement of metabolism via reduction of hepatic citrate uptake. In this study the inhibition of mINDY recapitulates the main features previously reported for mINDY knockout mice specifically reduction in hepatic lipid production and in plasma glucose levels following oral glucose tolerance test. It has been shown that inhibition of mINDY simultaneously reduce hepatic glucose and lipid production. However, based on the chemical properties and the resulting very high effective doses this molecule can be only considered as a tool compound but not drug like molecule for therapeutic treatment of metabolic diseases

Functionally, loss of mIndy also mimics many aspects of calorically restriction. Moreover, in flies and nematodes, both, reduced expression of Indy, as well as caloric restriction, prolong life span (Rogina et al, 2000, Science 290, p. 2137-2140; Fei et al, 2004, Biochem. J. 379, p. 191-198) and AMPK has been shown to be the mediator of longevity in response to most dietary restriction regimens in C. elegans (Schulz et al, 2007, Cell Metab 6, p. 280-293; Greer et al, 2009, Aging Cell 8, p. 113-127; Mair et al, 2011, Nature 470, p. 404-408). In addition, caloric restriction does not increase life span further in flies with reduced Indy expression (Toivonen et al, 2007, PLoS. Genet. 3, e95; Wang et al, 2009, Proc. Natl. Acad. Sci. U.S.A 106, p. 9262-9267), pointing to similar underlying mechanisms in both conditions. In fact, a very recent work by Schwarz and colleagues (Schwarz et al. 2015 Aging 7(8), p. 553-67) has been shown that knockdown of Indy in c. elegans extends life span by inducing AMPK, whereas the effect was abolished in worms without functional AMPK. This indicates that the life extending effect of reducing mINDY is at least in part mediated by AMPK. These data suggest that mIndy may be a key mediator of the beneficial effects of dietary energy restriction. Since prolonged caloric restriction is very difficult to achieve in humans, the observations raise the tantalizing possibility that modulating the levels or function of mIndy could lead to some of the health promoting effects of calorie restriction, without requiring severe caloric restriction.

Very recent work by De Costa Goncalves and colleagues showed that loss of mIndy also affects blood pressure (De Costa Goncalves Clin AutonRes 2014, 24:199-243 abstract 40). Deletion of mINDY reduces blood pressure in animals fed a HFD. These finding raises the possibility that mINDY is part of the signalling pathway linking excessive caloric intake to increased blood pressure. Therefore, it seems plausible to speculate that mINDY might be an interesting target for the treatment of hypertension.

Another recent work by Li and colleagues showing the link between mINDY and drug induced hepatic steatosis (Li et al. 2015 Mol Pharmacol. 87(4):674-82). Knockdown of INDY by antisense oligonucleotides leads to significant decrease of rifampicin induced lipid accumulation in HepG2 via PXR dependent pathway. These data were confirmed by Neuschafer-Rube et al. (Neuschafer-Rube et al. 2015 Toxicology 337, p. 1-9). This work showed the link between mINDY and drug induced hepatic steatosis in vitro by benzo[a]pyrene induced lipid accumulation in primary rat hepatocytes (via arylhyrocarbon receptor). Therefore, mINDY is an interesting target for the treatment of drug induced hepatic steatosis.

Importantly, all known studies to reduce INDY expression in vivo in mice as well as rats showed consistently a reduction of liver fat accumulation in animals under high fat diet (Birkenfeld et al, 2011, Cell Metab 14, p. 184-195, Pesta et al. 2015 Aging 7(12), p. 1086-93, Rong et al. 2015 Conference abstract Keystone Symposia: Obesity and the Metabolic Syndrome/Liver Metabolism March 2015, Huard et al. 2015 Sci Rep. 5, p. 17391, Willmes 2016 Aging 2, p. 208-9) further supporting the hypothesis that mINDY is a drug target for the treatment of non-alcoholic fatty liver disease (NAFLD) and non-alcoholic steatohepatitis (NASH).

To date, no pharmaceutically effective and specific therapeutic agents with drug-like properties to modulate mINDY function or expression are known, except an antisense oligonucleotide as tool compound. Interestingly, the stimulation of human mINDY activity by Lithium was described in concentrations that are observed during the treatment of bipolar disorders. Aluvila and colleagues disclosed compounds, which inhibit another but not mINDY related citrate transporter, the citrate transport protein (CTP) on the inner mitochondrial membrane (Aluvila et al, 2010, Mol Pharmacol 77, p. 26-34; Irwin and Shoichet, 2005, J Chem Inf Model 45, p. 177-182). Moreover, a compound with selectivity for mINDY over CTP was identified by Sun and co-workers (Sun et al. 2010, Mol Cell Pharmacol 2, p. 101-110). This compound shows inhibitory activity in a millimolar range in a cell free assay. However, in a cellular citrate uptake assay this compound seems to activate Indy.

Pajor and colleagues disclosed an inhibitor of Indy in a high micromolar range (Pajor et al. Mol Pharmacol. 2007 November; 72(5), p. 1330-1336). But it seems that this compound activity is related to cytotoxic side effects. Furthermore, Ganapathy and colleagues disclosed a substrate analogue, hydroxycitrate, as an inhibitor of Indy in a high micromolar range (30-40% inhibition at 0.1 mM) (WO 2004/048925). However, in a cellular citrate uptake assay it was not possible to reproduce this data. A recent work by Colas and Co-workers (Colas 2015 Biochemistry 54(31), p. 4900-8) used a combined modelling and virtual screening approach to find mINDY inhibitors. One active compound has been found with an activity in mM range in a vitro assay. Finally, Huard et al. (Huard et al. 2015 Sci Rep. 5, p. 17391) described a tool compound which was selective for mINDY with submicromolar activities in vitro. However, based on its characteristics and chemical properties very high doses (250 mg/kg bi-daily) are necessary to show a therapeutic effect in vivo. Therefore, this substrate analogue compound class seems to be not a suitable drug like molecule.

It is not known so far, whether such compounds can be used in therapeutic intervention. The high concentration needed to inhibit mINDY with these molecules or potential toxic side effects make it unlikely to become clinically relevant. The discovery of a more potent and specific compound modulating mINDY function could provide a useful tool to delineate the structure and function of mINDY and to become therapeutic drug to treat metabolic diseases. Ultimately, a putative inhibitor of mINDY holds the potential to induce the beneficial effects of caloric restriction, without requiring severe caloric restriction in mammals. (Willmes and Birkenfeld, 2013, Computational and Structural Biotechnology Journal. 6 (7))

Thus, there is a need for inhibitors of Indy in order to use such an inhibitor for the treatment of metabolic diseases. It is an object of the present invention to provide an inhibitor for the activity of Indy.

### BRIEF SUMMARY OF THE INVENTION

The present invention provides a compound for use in treating diseases depending on the activity of a citrate transporter, wherein the compound has the general formula (I)

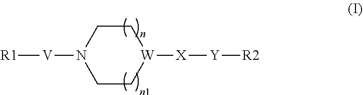

wherein R1 is Aryl, substituted aryl, styryl, or bicyclic; V is SO₂; W is N with n and n1 are 1 and X is C═O, or W is C with n and n1 are 0 or 1 and X is CH₂ or C═O, Y is N—H or N-methyl and R2 is benzyl, substituted benzyl or CH₂(2-pyridyl) with the proviso that the compound of formula (I) is not the following compound:

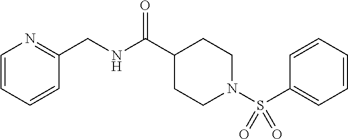 [SMILES: O=C(NCc1ccccn1)C1CCN(S(=O)(=O)c2ccccc2)CC1]

In one embodiment the compound is 1-(2-Phenyl-ethenesulfonyl)-azetidine-3-carboxylic acid 2-fluoro-benzylamide.

It is further envisaged that the citrate transporter is the gene product of Indy or a homologue thereof. The term “homologues” used in this disclosure refers to genes or proteins having similar or identical biological functions. The similarity or identity of the biological functions can be reflected by sequence similarity or identity (at either the amino acid or nucleotide level) of about 45%, about 70% or about 90%. Sequence similarity or identity (at either the amino acid or nucleotide level) within defined regions of the molecule or across the full-length sequence can be determined through sequence alignments using computer software programs such as BLAST, ALIGN, DNAstar and INHERIT which employ various algorithms to measure homology. A person skilled in the art is familiar with these alignment programs. Sequence regions that are homologous may be called conserved, consensus or canonical sequences and represent the most common choice of base or amino acid at each position.

The compound of general formula (I) binds to citrate transporter and influences their activity. Thus, it is within the scope of the present invention that the compound of general formula (I) influences the activity of the citrate transporter directly or allosterically. Allosteric inhibition or regulation means the regulation of a proteins or enzymes activity by binding to allosteric sites that are different from the active sites of the respective protein or enzyme.

Another object of the instant disclosure is 1-(2-Phenyl-ethenesulfonyl)-azetidine-3-carboxylic acid 2-fluoro-benzylamide as compound.

Further, a compound as mentioned above including pharmaceutically applicable salts, tautomers and stereoisomers of the compound, including mixtures thereof in all ratios for use in the treatment and/or prevention of diseases depending on the activity of a citrate transporter is an object of the instant disclosure.

Another object of the present invention is a compound of general formula (I) for the treatment and/or prevention of
a. metabolic diseases selected from the group comprising insulin resistance, alcoholic and non-alcoholic fatty liver disease, non-alcoholic steatohepatitis (NASH), obesity, type 1 diabetes, type 2 diabetes, dyslipidemia, hereditary diseases and metabolic syndrome, and
b. eating disorders, and
c. chronic liver diseases, and
d. liver cancer and cancer related to obesity.

Further a use of the compound of general formula (I) for extending life span by itself or by treatment and/or prevention of age-related diseases comprising atherosclerosis and cardiovascular disease, cancer, arthritis, cataracts, osteoporosis, type 2 diabetes, hypertension and neurodegenerative diseases like Alzheimer's disease is intended.

Another object of the present invention is the use of a compound of general formula (I) for preparing a medicament for the treatment and/or prevention of
a. metabolic diseases selected from the group comprising insulin resistance, alcoholic and non-alcoholic fatty liver disease, non-alcoholic steatohepatitis (NASH), obesity, type 1 diabetes, type 2 diabetes, dyslipidemia, hereditary diseases and metabolic syndrome, and
b. eating disorders, and
c. chronic liver diseases, and
d. liver cancer and cancer related to obesity and
e. age related diseases comprising atherosclerosis and cardiovascular disease, cancer, arthritis, cataracts, osteoporosis, type 2 diabetes, hypertension and neurodegenerative diseases like Alzheimer's disease.

Further, it is an object of the present invention to provide a medicament comprising a compound of general formula (I) alone or in combination with one or more further active compounds selected from the group comprising
a. anti-obesity agents selected from the group consisting of orlistat, lorcaserin, Phentermine, Topiramate, sibutramine, bromocriptine, ephedrine, leptin, and pseudoephedrine, 5-HT2c receptor agonists, Bupropion, Naltrexone, methionine aminopeptidase 2 inhibitors,
b. anti-diabetes agents comprising insulin, incretin mimetics, SGLT-2 inhibitors, DPPIV inhibitors, PPAR agonist, Glucokinase activator, MTP inhibitors, Glycogen phosphorylase inhibitors, DGAT-1 inhibitor,
c. anti-NASH agents comprising insulin, incretin mimetics, statins, PPAR agonists, AMPK activators, FXR agonists, DGAT-1 inhibitors, Bile-Acid Conjugates, methionine aminopeptidase 2 inhibitors, PDE4 inhibitors,
d. anti-dyslipidaemia agents comprising, statins, ApoB antisense oligonucleotides, PCSK9 inhibitors, Cholesterol-absorption inhibitors, Niacin, Bile-acid-sequestering resins, MTP inhibitors, Fibrates, CETP inhibitors,
e. anti-cancer agents comprising chemotherapeutic drugs and
f. anti aging drugs comprising vitamins.

It is intended that a medicament of the invention is suitable for the treatment and/or prevention of
a. metabolic diseases selected from the group comprising insulin resistance, alcoholic and non-alcoholic fatty liver disease, non-alcoholic steatohepatitis (NASH), obesity, type 1 diabetes, type 2 diabetes, dyslipidemia, hereditary diseases and metabolic syndrome, and
b. eating disorders, and
c. chronic liver diseases, and
d. liver cancer and cancer related to obesity and
e. age related diseases comprising atherosclerosis and cardiovascular disease, cancer, arthritis, cataracts, osteoporosis, type 2 diabetes, hypertension and neurodegenerative diseases like Alzheimer's disease.

A further object of the present invention is the use of a compound of general formula (I) for diagnosis of
a. metabolic diseases selected from the group comprising insulin resistance, alcoholic and non-alcoholic fatty liver disease, non-alcoholic steatohepatitis (NASH), obesity, type 1 diabetes, type 2 diabetes, dyslipidemia, hereditary, diseases and metabolic syndrome, and
b. eating disorders, and
c. chronic liver diseases, and
d. liver cancer and cancer related to obesity and
e. age related diseases.

It is further intended that the compound of general formula (I) is linked to functional moieties for its use in detection assays comprising radio nucleotides, fluorophores and enzymes.

### DETAILED DESCRIPTION OF THE INVENTION

The presentment invention provides an inhibitor for citrate transporters like the gene product of Indy.

In one aspect the invention is directed to novel compounds. Further, the invention is directed to the use of compounds as disclosed as a medicament.

One embodiment of the invention is directed to compositions and methods for treating a metabolic disorder in a subject such as obesity, hyperglycemia, alcoholic and non-alcoholic fatty liver disease (NAFLD), non-alcoholic steatohepatitis (NASH), type 1 diabetes, type 2 diabetes, dyslipidemia, inflammatory diseases caused by adiposity, and cancers associated with obesity.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. In certain embodiments a therapeutically effective amount may achieve one or more of lowering blood glucose level, decreasing insulin resistance and increasing insulin sensitivity, lowering hepatic lipids, lowering hepatic triglycerides, lowering hepatic diacylglycerol, lowering blood cholesterol, lowering blood triglycerides, lowering blood LDL, lowering muscle diacylglycerols.

Diabetes Mellitus generally refers to fasting plasma glucose values of >126 mg/dL (>7.0 mmol/i) and insulin resistance is defined here as a fasting blood insulin level greater than 20 mcU/mL. Adiposity and obesity both refer to a medical condition in which excess body fat has accumulated to an extent where it may increases the likelihood of various diseases, particularly heart disease, type 2 diabetes, obstructive sleep apnoea, certain types of cancer, and osteoarthritis. Generally adiposity and obesity are related to a Body Mass Index (kg/m²) above 25. NAFLD refers to a wide spectrum of liver clinicopathologic conditions, ranging from pure fatty steatosis (fatty infiltration in >5% of hepatocytes) to nonalcoholic steatohepatitis (NASH), which may progress to cirrhosis, liver failure, and hepatocellular carcinoma and is characterized by excessive fat accumulation in the liver parenchyma of patients who have no history of alcohol abuse. NASH refers to a medical condition with presence of specific histological abnormalities on liver biopsy such as a characteristic pattern of steatosis, inflammation and hepatocellular ballooning in the absence of significant alcohol consumption.

Age-related diseases occur with increasing frequency with increasing senescence such as atherosclerosis and cardiovascular disease, cancer, arthritis, cataracts, osteoporosis, type 2 diabetes, hypertension and neurodegenerative diseases like Alzheimer's disease. The incidence of all of these diseases increases rapidly with aging. One aspect of age related diseases concerns pre-diabetes, a condition that raises a person's risk for developing type 2 diabetes, heart disease, and stroke. Within the context of the present invention such diseases shall be understood as age related diseases.

Potential cancers to be treated with a compound of general formula (I) comprise liver, pancreas cancer, breast cancer, oesophagus cancer, pancreas cancer, colon cancer, gallbladder cancer, colorectal cancer, endornetrium cancer, kidney cancer, gallbladder cancer, thyroid cancer, rectal cancer, melanoma, leukaemia, multiple myeloma, non-Hodgkin lymphoma, prostate cancer, uterine cancer, ovarian cancer, endometrial cancer and cervical cancer.

The compound represents a basis for further development and modification of the basic formula. Within the scope of the present invention are pharmaceutically acceptable salts, prodrugs, enantiomers, diastereomers, racemic mixtures, crystalline forms, non-crystalline forms, amorphous forms, unsolvated forms and solvates compound of the general formula (I).

The term “pharmaceutically acceptable salts” as used herein includes salts of the compound of the general formula (I) which are prepared with relatively nontoxic (i.e. pharmaceutically acceptable) acids or bases, depending on the particular substituents found on the compounds of the present invention. If, for example, compounds of the present invention contain acidic functionalities, base addition salts may be obtained by contacting the neutral form of such compounds with a sufficient amount of the desired base, either neat or in a suitable inert solvent. Non-limiting examples of pharmaceutically acceptable base addition salts include sodium, potassium, calcium, ammonium, organic amino, or magnesium salt, or a similar salt. If compounds of the present invention contain basic functionalities, acid addition salts may be obtained by contacting the neutral form of such compounds with a sufficient amount of the desired acid, either neat or in a suitable inert solvent. Non-limiting examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic. malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention may contain both basic and acidic functionalities that allow the compounds to be converted into either base or acid addition salts. Contacting the salt with a base may regenerate the neutral forms of the compounds of the present invention or acid and isolating the parent compound in the conventional manner. The parent form of the compound differs from the various salt forms in certain physical properties, such as solubility in polar solvents, but otherwise the salts are equivalent to the parent form of the compound for the purposes of the present invention. The compounds of the present invention may possess chiral or asymmetric carbon atoms (optical centers) and/or double bonds. The racemates, diastereomers, geometric isomers and individual optical isomers are encompassed by the present invention. The compounds of the present invention may exist in unsolvated forms as well as solvated forms, including hydrated forms. In general, the solvated forms are equivalent to unsolvated forms and are also encompassed by the present invention. The compounds of the present invention may furthermore exist in multiple crystalline or amorphous forms.

The compounds of the present invention may further be in a so-called prodrug form. Prodrugs of the compounds of the invention are those compounds that readily undergo chemical changes under physiological conditions to provide the compounds of the present invention. Additionally, prodrugs can be converted to the compounds of the present invention by chemical or biochemical methods in an ex-vivo environment. For example, prodrugs can be slowly converted to the compounds of the present invention when, for example, placed in a transdermal patch reservoir with a suitable enzyme or chemical reagent.

The compound of the invention described herein can be administered to the subject at a suitable dose. The compound of the invention is preferably administered to mammals such as domestic and pet animals. Non-limiting examples of domestic and pet animals are pigs, cows, buffalos, sheep, goats, rabbits, horses, donkeys, chickens, ducks, cats, dogs, genuine pigs, or hamsters. Most preferred it is administered to humans. The preferred way of administration depends on the form of the compound of the invention (having the general formula (I)). As described herein above, the compound having the general formula (I) can be in the form of pharmaceutically acceptable salts, prodrugs, enantiomers, diastereomers, racemic mixtures, crystalline forms, non-crystalline forms, amorphous forms, unsolvated forms or solvates. The compound of the invention may be administered orally, parenterally, such as subcutaneously, intravenously, intramuscularly, intraperitoneally, intrathecally, intraocular, transdermally, transmucosally, subdurally, locally or topically via iontopheresis, sublingually, by inhalation spray, aerosol or rectally and the like in dosage unit formulations optionally further comprising conventional pharmaceutically acceptable excipients. The compound of the invention for use in accordance with the present invention can be formulated as a pharmaceutical composition using one or more physiological carriers or excipient, see, for example Ansel et al., “Pharmaceutical Dosage Forms and Drug Delivery Systems”, 7th edition, Lippincott Williams & Wilkins Publishers, 1999.

For oral administration, the pharmaceutical composition of the invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). The pharmaceutical composition can be administered with a physiologically acceptable carrier to a patient. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can be in the form of ointments, solutions, suspensions, emulsion, tablets, pills, capsules, powders, sustained-release formulations and the like. A preferred form is an ointment. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. E. W. Martin describes examples of suitable pharmaceutical carriers in “Remington's Pharmaceutical Sciences”. Such compositions will contain a therapeutically effective amount of the aforementioned compounds, preferably in purified form, together with a suitable amount of carrier so as to provide the form for proper administration to the patient. The formulation should suit the mode of administration. Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, soric acids). The preparations can also contain buffer salts, flavouring, coloring and sweetening agents as deemed appropriate. Preparations for oral administration can be suitably formulated to give controlled release of the pharmaceutical composition of the invention.

For administration by inhalation, the pharmaceutical composition of the invention is conveniently delivered in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide or other suitable gas). In the case of a pressurised aerosol, the dosage unit can be determined by providing a valve to deliver a metered amount. Capsules and cartridges of, for example, gelatine, for use in an inhaler or insufflator can be formulated containing a powder mix of the pharmaceutical composition of the invention and a suitable powder base such as lactose or starch.

The pharmaceutical composition of the invention can be formulated for parenteral administration by injection, for example, by bolus injection or continuous infusion. Site of injections include intra-venous, intra-peritoneal or sub-cutaneous. Formulations for injection can be presented in units dosage form (e.g., in phial, in multi-dose container), and with an added preservative. The pharmaceutical composition of the invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anaesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

It is obvious for a person ordinary skilled in the art that the present invention also encompasses sustained release dosage forms, which are designed to release a drug at a predetermined rate in order to maintain a constant drug concentration for a specific time period of time with minimum side effects. This can be achieved through a variety of formulations or devices, including microspheres, nanoparticles, liposomes, and other polymer matrices such as drug-polymer conjugates like hydrogels or biodegradables like poly(lactic-co-glycolic acid) (PLGA) encapsulating the drug. It is preferred to adapt the release to the specific needs for treating particular diseases, e.g. like sustained release of injections in treating diabetes. Sustained release's definition is more akin to a “controlled release” or “depot medication” rather than “sustained”.

The pharmaceutical composition of the invention can also, if desired, be presented in a pack, or dispenser, which can contain one or more unit dosage forms containing the said agent. The pack can for example comprise metal or plastic foil, such as blister pack. The pack or dispenser device can be accompanied with instruction for administration.

The pharmaceutical composition of the invention can be administered as sole active agent or can be administered in combination with other active agents. Such additional active agents should be primarily chosen from active agents being related to the treatment of the same disease. In case that obesity shall be treated an additional active agent should be chosen from the group of anti-obesity drugs. In analogy anti-diabetes and also anti-NAFLD/NASH as well as anti-dyslipidaemia drugs may be used as further active agents. Furthermore, such additional active agent should be chosen from active agents being related to side effects such as body weight gain like anti-psychotic treatments.

For obesity combinations may comprise combination therapies that are administered in conjunction with exercise, combination therapies that are administered in conjunction with sensible diet, combination therapies with anti-obesity agents are selected from the group consisting of orlistat, lorcaserin, Phentermine, Topiramate, sibutramine, bromocriptine, ephedrine, leptin, and pseudoephedrine. Further examples of combinations with a compound of general formula (I) are lipase inhibitors (e.g. Orlistat Xenical®, Roche, Alli®, GSK, Cetilistat), 5-HT2c receptor agonists (e.g. Lorcaserin, Belviq® Arena Inc., Eisai), phentermine and topiramate (e.g. Qsymia®, Vivus Inc), noradrenergic anorectic agents (e.g. phentermine, mazindol), appetite suppressants (for example, bupropion), bupropion and Naltrexone (e.g. Contrave®, Orexigen Inc.), drugs affecting endogenous signaling of appetite-regulating hormones, neuropeptides, neurotransmitters (e.g. targeting neuro peptide Y receptor, MOR, AgRP, MCHR 1, H3 R), human agouti-related proteins (AGRP), ghrelin receptor antagonists, histamine 3 receptor antagonists or reverse agonists, neuropeptide-Y antagonists, MCR-4 agonists, melanocyte-stimulating hormone receptor analogs, melanin concentrating hormone antagonists, apolipoprotein-B secretion/microsomal triglyceride transfer protein (apo-B/MTP) inhibitors (e.g. JNJ16269110, J&J), GR-II antagonist (e.g. CORT108297, Corcept Therapeutics Inc), GLP1 agonists (e.g. Exenatide (Byetta®, Ely Lilly), Liraglutide (Victoza®), Novo Nordisk), Glucokinase activator (e.g. AZD1656, AstraZeneca), SGLT-2 inhibitor (e.g. gliflozines such as Invokana™ (canagliflozin), J&J; remogliflozin, Kissei, GSK, Dapagliflozin (Forxiga®, BMS, Astra Zeneca)), PPAR alpha and -gamma agonist (e.g. glitazars such as LBM642 (cevoglitazar), Novartis, Aleglitazar, Roche), MetAP inhibitor (e.g.CKD732 (beloranib), Zafgen), cholescystokinin-A (CCK-A) agonists, serotonin and norepinephrine reuptake inhibitors (e.g. sibutramine), sympathomimetic agents, 03 adrenergic receptor agonists, dopamine agonists (e.g. bromocriptine), cannabinoid 1 receptor antagonists e.g. SR141716: N-(piperidin-1-yl)-5-(4-chlorophenyl)-1-(2,4-dichlorophenyl)-4-methyl-1H-pyrazole-3-carboxamide], leptons (the OB protein), leptin analogues, leptin receptor agonists, galanin antagonists, lipase inhibitors (such as tetrahydrolipstatin, i.e., Orlistat), anorectic agents (such as a bombesin agonist), thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™ Regeneron Pharmaceuticals) and neuromedin U receptor agonists.

For T2DM combinations may be selected from Incretin mimetics, GLP1 agonists (e.g. Exenatide (Byetta®, Ely Lilly), Liraglutide (Victoza®), Novo Nordisk), GPR119 agonist (e.g. PSN-821, Astra Zeneca), GPR40 agonist (e.g. Fasiglifam, Takeda, ASP5034, Astellas), SGLT-2 inhibitor (e.g. gliflozines such as Dapagliflozin (Forxiga®, BMS, Astra Zeneca), Canagliflozin (Ivokana® J&J)), DPPIV inhibitors (e.g. gliptine such as Sitagliptin (Januvia®, Merck)), PPAR agonist (e.g. glitazones such as Rosiglitazone (Avandia®), GSK), Dual PPAR alpha and -gamma agonists (e.g. glitazars such as Cevoglitazar, Novartis, Aleglitazar, Roche)), Glucokinase activator (e.g. AZD 1656, AstraZeneca), MTP inhibitors (e.g. JNJ16269110, J&J), Glycogen phosphorylase inhibitor, 11-beta-HSD-1(e.g. INCB13739 Incyte, AZD4017 AZ), DGAT-1 inhibitor, aldose reductase inhibitor, a sorbitol dehydrogenase inhibitor, a protein tyrosine phosphatase lB inhibitor, an insulin mimetic, metformin, acarbose, a sulfonylurea, glipazide, glyburide, or chlorpropamide, glucosidase inhibitor, meglitimide and an αP2 inhibitor.

For NAFLD/NASH combinations may comprise incretin mimetics, GLP1 agonists (e.g. Exenatide (Byetta®, Ely Lilly), Liraglutide (Victoza®), Novo Nordisk), GPR119 agonist (e.g. PSN-821, Astra Zeneca), GPR40 agonist (e.g. Fasiglifam, Takeda, ASP5034, Astellas), statins (HMG-CoA Reductase, e.g. atorvastatin (Lipitor), fluvastatin (Lescol), lovastatin (Mevacor, Altocor), pitavastatin (Livalo), pravastatin (Pravachol), rosuvastatin (Crestor) and simvastatin (Zocor)), PPARgamma agonists (e.g. Pioglitazone), AMPK (e.g. Metformin), Drugs which increase cholesterol secretion into bile by synthetic Fatty-Acid/Bile-Acid Conjugates (e.g. Aramchol (Galmed)), FXR agonist (e.g. INT747 (Intercept Pharm.), Px-102/104 (Phenex)), Cysteamine bitartrate (adiponectin multimerization) (e.g. RP103 (Raptor Pharm)), DGAT-1 inhibitor (e.g. LCQ908 (Novartis)), SAMe (methionine metabolism), oral (anti-CD3 antibody) (e.g. OKT3 (NasVax)), LOXL2 Mab (e.g. Simtuzumab (Gilead)), Omega-3 fatty acid (e.g. EPA-E (Moichida Pharm)), Dual PPARa/d agonist GFT505 (GENFIT), PDE4 inhibitor (e.g. Roflumilast (Takeda)), immunomodulation by glucospingolipid (e.g. EGS21 (Enzo)).

For hyper/dyslipidemia combinations may comprise statins (e.g. HMG-CoA reductase inhibitor such as atorvastatin (Lipitor), fluvastatin (Lescol), lovastatin (Mevacor, Altocor), pitavastatin (Livalo), pravastatin (Pravachol), rosuvastatin (Crestor) and simvastatin (Zocor), ApoB antisense oligonucleotides (e.g. mipomersen, Kynamro), PCSK9 inhibitors (e.g. antibodies such as AMG145 (Amgen), 1D05-IgG2 (Merck & Co.), and SAR236553/REGN727 (Aventis/Regeneron) or antisense RNA such as ALN-PCS, Amgen), cholesterol-absorption inhibitors (e.g. NPC1L1 inhibitors such as ezetimibe), niacin, bile-acid-sequestering resins (e.g. Cholestyramine (Questran), Colesevelam (Cholestagel, Welchol), Colestipol (Colestid) Colestipid, MTP inhibitors (e.g. lomitapide), fibrates (e.g. Bezafibrate (e.g. Bezalip), Ciprofibrate (e.g. Modalim), Clofibrate, Gemfibrozil (e.g. Lopid), Fenofibrate (e.g. TriCor)), CETP inhibitors (e.g. dalcetrapib, torcetrapib anacetrapib and evacetrapib).

For anti-psychotic treatment combinations may comprise Butyrophenones (e.g. Haloperidol), Diphenylbutylpiperidine (e.g. Fluspirilene, Penfluridol, Pimozide), Phenothiazines (e.g. Fluphenazine Perazine Perphenazine Promethazine Trifluoperazine), Thioxanthenes (e.g. Clopenthixol Tiotixene) or Clozapine, Olanzapine, quetiapine, zotepine).

### EXPERIMENTAL PROCEDURES

The invention will be described by experimental procedures without being limited to the disclosed embodiments.

The compound was obtained by screening of a library containing more than 50,000 lead-like compounds and fragments. The library was tested using a functional citrate uptake assay with HEK293 cells overexpressing recombinant human INDY. The following hit confirmation was conducted in the same assay system. To confirm the activity of selected compounds on the target INDY another cell assay was conducted. In this assay system citrate uptake was measured using HepG2 cells which endogenously express INDY. Furthermore selectivity of selected compounds was tested for two other transporters in different assay systems. The first transporter was the glutamate transporter GLT-1 tested in a glutamate uptake assays using HEK293 cells overexpressing recombinant human GLT-1. The second transporter was the glucose transporter GLUT1 tested in a glucose uptake assay using Huh-7 cells which endogenously express GLUT1. Compounds were considered to be selective with an at least 5-fold higher activity for INDY than for the other two transporters.

Cell cultivation media and reagents were obtained from standard suppliers. Cultivation of cells was done as known by a person ordinary skilled in the art (comp. Green and Sambrook, Molecular Cloning: A laboratory handbook, 4^{th} Edition 2012, Cold Spring Harbour Laboratory Press).

Cells were maintained in cell medium using cell culture grade flasks (CellSTACK Corning, 500 cm2 dishes or T175 depending on cell number). The selection antibiotic and G418 (800 μg/ml) was added during cultivation but not for seeding into assay plates. For splitting, cells were washed with PBS (w/o Ca2+, Mg2+, phenol red) and detached with Trypsin/EDTA. Throughout cultivation cells were kept sub-confluent. Detached cells (50,000 cells/well) were seeded in 96-well plates (white, clear bottom from Corning #655098) and incubated over night before usage in the assay. The following media were used. For HEK293 cells expressing INDY MEM (PAA)+10% FCS+1×P/S+2 mM L-Gln; for HEK293 cells expressing GLT-1 DMEM (Gibco)+10% FCS+400 g/ml G418; for Huh7 cells DMEM (Gibco)+10% FCS+2 mM L-Gin (+1×P/S); for HepG2 cells MEM (PAA, Cat # E15-024)+10% FCS+2 mM L-Gin+1×NEAA+1 mM Na-Pyruvat

The following assay buffers for the particular uptake reaction were used. Citrate uptake buffer: 120 mM NaCl, 5.4 mM KCl, 0.8 mM MgSO₄, 5 mM glucose, 1.8 mM CaCl₂, 25 mM Hepes, 25 mM MES, pH 6.5. Glutamate uptake buffer: HBSS, 1 mM CaCl₂, 1 mM MgCl₂, 20 mM Hepes pH 7.4. Glucose uptake buffer: 25 mM Hepes+25 mM MES pH 7.4, 120 mM NaCl, 5.4 mM KCl, 1.8 mM CaCl₂, 0.8 mM MgSO₄.

INDY mediated citrate uptake was determined in HEK293 cells. To determine the uptake of ¹⁴C labelled citrate into cells, HEK293 cells over-expressing human NaCT were used. Cloning of human NaCT was done in analogy as described in Birkenfeld et al. (Birkenfeld et al., 2011, Cell Metabolism 14, 184-195, 2011).

Human NaCT HEK293 cells were seeded into white clear-bottom 96-well plates (50,000/well) in the presence of 4 μg/ml Poly-D-Lysine and incubated at 37° C. for 16-24 hours. On the days of the assay the plates were washed once with assay buffer using automated washing/LS 405 Selectors, BioTek ending with a buffer volume of 40 μl. Then 5 μl of compound solution in assay buffer was added and incubated for 5-30 min at 37° C. in cell incubator, HERA cell Heraeus. Afterwards 10 μl of substrate 14C-citrate (1.5 nM/8.7.4 nCi per well) were added and incubated for another 20-90 min at 37° C. in cell incubator, HERAcell. Then plates were washed twice with 200 μl ice-cold assay buffer and residual volume was discarded. For cell lysis 50 μl ice-cold NaOH (100 mM) was added and incubated for 15 minutes at RT on a plate shaker. Finally, 200 μl scintillation fluid (scintillator OptiPhase Supermix, Perkin Elmer) was added. After 15 min incubation at RT without shaking, plates were measured using a TopCount reader system (Perkin Elmer). As a positive control 10 mM citrate was applied. It is possible to use this assay also in analogy in a 384-well plate format.

INDY mediated citrate uptake was also determined in HepG2 cells. To determine the uptake of ¹⁴C labelled citrate into cells, HepG2 cells endogenously expressing human NaCT were used. (Gopal et al. 2007 Am J Physiol Gastrointest Liver Physiol 292).

HepG2 cells were seeded into white clear-bottom 96-well plates (50,000/well) coated with collagen and incubated at 37° C. for 16-24 hours. After that plates were washed once with assay buffer using automated washing/LS 405 Selectors, BioTek ending with a buffer volume of 40 μl. Then 5 μl of compound solution in assay buffer was added and incubated for 5 min at 37° C. in cell incubator, HERA cell Heraeus. Afterwards 10 μl of substrate 14C-citrate (10 nM) were added and incubated for another 90 min at 37° C. in cell incubator, HERAcell. Then plates were washed 3× with 200 μl buffer and 60 μl cell lysis induced by adding 50 μl ice-cold NaOH (100 mM). Plates were incubated for 25 min at RT on a plate shaker and afterwards 200 μl scintillation fluid was added and incubated for 15 min on a plate shaker at RT and for another 15 min without shaking at RT. Plates were measured using a TopCount system. As a positive control 5 mM citrate was applied. It is possible to use this assay also in analogy in a 384-well plate format.

As a counter assay, Glutamate uptake by the GLT-1 transporter was used. The principle of the assay is to measure the uptake of ³H labelled glutamate into HEK cells, which overexpress human GLT-1.

The generation of a GLT-1 cell line was performed as follows: human GLT11 isoform sv1 cDNA (Origene # RC223924) was cloned into vector pFB-Neo-CMV-hGLT1 using QIAfilter Plasmid Midi Kit (Qiagen). Virus was generated in GP293 packaging cells (transfection of plasmid with Lipofectamine 2000+OptiMEM). For transfection into HEK cells GP293, supernatant containing virus was passed through a 0.45 micron sterile filter and DEAE-dextran was added at 10 mg/ml final concentration. The solution was used to transfect HEK293 cells at 37° C., 8.5% CO₂. After 24 h selection marker G418 at 400 μg/ml was added and cultivation medium performed in DMEM 10% FBS.

The assay was performed in a 96-well format using repeated washing steps, cell lysis and addition of scintillation fluid. Assay buffer was HBSS, 1 mM CaCl₂, 1 mM MgCl₂, 25 mM Hepes pH 7.4. Cells were seeded into white clear-bottom 96-well plates (50,000/well) in the presence of Poly-D-Lysine and incubated at 37° C. for 16-24 hours. Subsequently the plates were washed once with assay buffer using automated washing/LS 405 Selectors, BioTek ending with a buffer volume of 40 μl. Then 5 μl of compound solution in assay buffer was added and incubated for 20 min at 37° C. in cell incubator, HERA cell Heraeus. Afterwards 10 μl of substrate 3H-Glu (3 nM) were added and incubated for another 20 min at 37° C. in cell incubator, HERAcell. Then the plates were washed once with 200 μl cold assay buffer and 150 μl scintillation fluid was added. Then plates were shaked for 30 min and incubated for another 15 min without shaker, Titramax 1000, Heidolph and finally measured using Top count, Perkin Elmer. As a positive control 1 mM glutamate was applied.

Glucose uptake in Huh7 cells was measured as a counter assay. The principle of this assay is to determine the uptake of ³H labelled glucose into Huh7 cells. Hepatocellular cell lines like Huh-7 cells overexpress endogenously several glucose transporters from the GLUT family mainly GLUT1 (Brito et al. EASL 2011, Amman et al. 2009 Am J Pathol 174(4)). As assay buffer 25 mM Hepes+25 mM MES pH 7.4, 120 mM NaCl, 5.4 mM KCl, 1.8 mM CaCl₂, 0.8 mM MgSO₄ was used. Huh7 cells were seeded into collagen-coated 96-well plates (white, clear bottom; 50,000/well) and incubated at 37° C. for 16-24 hours. The plate were washed once with assay buffer using automated washing/LS 405 Selectors, BioTek ending with 40 μl buffer followed by adding 5 μl of compound solution in assay buffer and incubation for 5 min at 37° C. in a cell incubator, HERA cell Heraeus. Afterwards 10 μl of substrate 3H-glucose (0.113 nM; 100 nCi/well) were added and incubated for another 30 min at 37° C. in ca ell incubator, HERAcell. The plate were washed twice with 200 μl buffer. Cell lysis was induced by adding 50 μl ice-cold NaOH (100 mM). Plates were incubated for 20 min at RT and afterwards 200 μl scintillation fluid added. After 20 min incubation at RT plates were measured using TopCount system, Perkin Elmer. 100 μM phloretin was added as positive control.

In order to identify compounds with potential cytotoxic properties, cell imaging was used. Alternatively, cell protein content was determined.

For imaging, cells were stained with propidium iodide (PI) which does not cross the cellular membrane and therefore only stains the nucleus if pores are present indicating late apoptosis. In parallel, a Hoechst stain was applied which can penetrate the cell membrane and therefore stains all cell nuclei. Early apoptotic effects are identified by condensed and rounded nuclei.

The corresponding cells (depends on assay) were seeded into imaging-compatible black clear-bottom 384-well plates (Greiner #781956, black μclear, collagen coated for HepG2 or Huh7 or Corning #3683BC for HEK cell lines in the presence of Poly-D-Lysine) (20,000/well) and incubated at 37° C. for 16-24 hours. Subsequently plates were washed twice using automated washing/LS 405 Selectors, BioTek ending with 25 μl of the corresponding assay buffer. Then 5 μl of compound solution in assay buffer was added and incubated for 2-24 hours at 37° C. for in cell incubator, HERA cell Heraeus. Optionally cells were fixed with 4% paraformaldehyde (PFA). Then of 45 μl propidium iodide (0.05 mg/ml) and Hoechst (1 μM) stain were added and incubated for 30 min at RT. Afterwards plates were analysed using the Opera™ High Content Screening system (Perkin Elmer). As a positive control 20 μM staurosporine was applied. The PI stain was analysed by counting the detected nuclei as an indicator for late toxicity. The Hoechst stain was analysed by combining the cell roundness factor and the detected cell area (Hoechst_readout=Cell_roundness/Cell_area).

For determination of the cell protein content, 10 μl of cell lysate was removed after lysis with 60 μl NaOH and transferred into a fresh assay plated into 15 μl PBS. Then, 200 μl Pierce BCA protein assay kit (Thermo Scientific) was added and incubated according to the supplier instructions. Absorbance at 562 nm was measured on a plate reader.

To test the functional activity of selected compounds and to bridge between the in vitro assay for citrate uptake to the physiological relevant lipid accumulation in liver cells a further functional cell assay was performed. This assay measured the effect of selected compounds on the citrate mediated fatty acid synthesis in hepatocytes.

Fatty acid synthesis (FAS) assay in HepG2: The principle of the assay is to measure the generation of ¹⁴C-labelled fatty acids after uptake of ¹⁴C labelled citrate into HepG2 cells. HepG2 cells were seeded into white clear-bottom 96-well plates (50,000/well) coated with collagen and incubated at 37° C. for 16-24 hours. After that plates were washed once with 100 μl PBS (+CaCl₂+MgCl₂) and 40 μl assay medium (RPMI 1860 containing 11 mM glucose, 10 mM HEPES and 1 nM Insulin) per well was added. Test substances in assay medium were added (51, 0.5% DMSO) and incubated for 20 min. After that 10 μl assay medium containing ¹⁴C-citrate (final concentration 50 nM) and PrestoBlue reagent (1× final concentration; Life Technologies) were added and incubated for 60 min (37° C., 5% CO₂). Cell viability was measured (Safire microplate reader, ex: 560/em: 590) and plates were incubated for another 30 min. Medium was removed by inverting the plate and cells were washed once with 100 μl ice-cold PBS. Cells were lysed with 50 μl lysis buffer (100 mM NaOH, 0.1% Triton X-100) and plate was sealed with Tape Pads and vortexed. For the saponification reaction plates were incubated for 16-24 hours at 80° C. Seals were removed and 200 μl 100 nM HCl was added for neutralisation of the pH. 150 μl from each well was transferred into the corresponding wells of a 96-well CytoStarT plate (Perkin Elmer) and sealed with TopSeal A (Perkin Elmer). Plates were incubated for 4 h at 70° C. and for 1 h at room temperature in the dark. Signal of ¹⁴C radioactivity was measured (TopCount NXT scintillation counter, 1 min/well). As a positive control 5 mM citrate or 40 μM C75 (Sigma, C5490) was applied.

Following table 1 shows the results of the assays:

| | Uptake of | | | | |
|---|---|---|---|---|---|
| | | | | | Citrate/ |
| | | | | | fatty acid |
| | citrate | citrate | glutamate | glucose | synthesis |
| | Transporter protein | | | | |
| | INDY | INDY | GLT-1 | GLUT1 | INDY |
| | Cell line | | | | |
| | HEK293 | HepG2 | HEK293 | Huh7 | HepG2 |
| Example | IC50 in μM | | | | |
| 1 | n.d. | 5.4 | n.d. | >50 | n.d. |
| 2 | n.d. | 4.1 | n.d. | n.d. | n.d. |
| 3 | n.d. | 11.3 | n.d. | n.d. | n.d. |
| 4 | n.d. | 5.7 | n.d. | n.d. | n.d. |
| 5 | 2.9 | 2 | >50 | >50 | 1.3 |
| 6 | n.d. | 5.7 | >50 | >50 | n.d. |
| 7 | n.d. | >50 | n.d. | n.d. | n.d. |
| 8 | n.d. | >50 | n.d. | n.d. | n.d. |
| 9 | n.d. | >50 | n.d. | n.d. | n.d. |
| 10 | n.d. | >50 | n.d. | n.d. | n.d. |

The solvents and column conditions used in the LC-MS analysis of the following examples are reported using methods (A), (B), (C) or (D). The respective method employed is recorded in the analytical LC-MS results.

TABLE 2
Method A and B

| Method | Method A (0990) MS10 | | Method B (MSQ1) | |
|---|---|---|---|---|
| HPLC | Agilent G1312A | | Shimadzu Prominence Series | |
| MS | Waters ZQ | | Waters SQ | |
| MSD Signal | Scan pos 100-1000 | | Scan pos 150-850 | |
| Settings | | | | |
| Column | Phenomenex Gemini C18 | | Phenomenex Part No. | |
| | 2.0 × 50 mm, 3 μm | | 00D-4498-AN | |
| | | | Kinetix-XB C18 | |
| | | | 100 × 2.1 mm, 1.7 μm | |
| Solvent (eluent) | | | | |
| A | A = 2 mM amm, bicarbonate, | | A = Formic acid (aq) 0.1% | |
| | buffered to pH 10 | | | |
| B | B = Acetonitile | | B = Formic acid | |
| | | | (acetonitrile) 0.1% | |
| Detection | | | | |
| Signal | UV215 | | UV215 | |
| Spectrum | range: 200-420 nm; | | range: 220-420 nm; | |
| | step: 1 nm | | step: 1 nm | |
| Injection | 3 | μl | 3 | μl |
| flow | 1 | ml/min | 0.6 | ml/min |
| Column temp. | 60° | C. | 40° | C. |

Pump gradient

pump gradient

| | Time (mins) | % Organic |
|---|---|---|
| Gradient | 0.00 | 1 |
| | 1.80 | 100 |
| | 2.10 | 100 |
| | 2.30 | 1 |
| | 3.50 | 1 |
| | 0 | 5 |
| | 5.30 | 100 |
| | 5.80 | 100 |
| | 5.82 | 5 |

TABLE 3
Method C and D:

| Method | Method C (1416) | | Method D (1673) | |
|---|---|---|---|---|
| HPLC | Shimadzu Prominence Series | | Shimadzu Prominence Series | |
| MS | Shimadzu LCMS-2010EV system | | Shimadzu LCMS-2010EV system | |
| MSD Signal | Scan pos 100-1000 | | Scan pos 100-1000 | |
| Settings | | | | |
| Column | Waters Atlantis dC18 | | Supelco Part No. | |
| | 2.1 × 100 mm, 3 μm | | 53802-U Supelco | |
| | | | Ascentis Express C18 | |
| | | | 30 × 2.1 mm, 2.7 μm | |
| Solvent (eluent) | | | | |
| A | A, Formic acid (aq) 0.1% | | | |
| B | B, Formic acid (CH3CN) 0.1% | | A = Formic acid (aq) 0.1% | |
| Detection | | | B = Formic acid | |
| | | | (acetonitrile) 0.1% | |
| Signal | UV 215 | | UV215 | |
| Spectrum | range: 210-420 nm; | | range: 220-420 nm; | |
| | step: 1 nm | | step: 1 nm | |
| Peak width | | | | |
| Injection | 3 | μl | 3 | μl |
| flow | 1 | ml/min | 1 | ml/min |
| Column temp. | 40° | C. | 40° | C. |

pump gradient

| | Time (mins) | % Organic |
|---|---|---|
| Gradient | 0.00 | 1 |
| | 1.80 | 100 |
| | 2.10 | 100 |
| | 2.30 | 1 |
| | 3.50 | 1 |
| | 0 | 5 |
| | 5.30 | 100 |
| | 5.80 | 100 |
| | 5.82 | 5 |

¹H NMR were run on either a Bruker Avance III HD 500 MHz or Bruker Avance III HD 250 MHz instrument. The solvents in the parentheses in NMR show the solvents used for the measurement. DMSO represents dimethylsulfoxide; CDCl₃ represents deuterated chloroform. The following abbreviations are used in reporting the ¹H NMR spectra: s (singlet), d (doublet), t (triplet), q (quartet).

Compounds used in the present specification were commonly named using a computer program capable of naming in accordance with IUPAC rules; JChem for Excel or MarvinSketch manufactured by ChemAxon Ltd.

### Abbreviations

DCM dichloromethane
DIPEA diisopropylethylamine
AcOH acetic acid
EtOAc ethyl acetate
MeOH methanol
Na₂SO₄ sodium sulphate
MgSO₄ magnesium sulphate
MeCN acetonitrile
TBME tert-butylmethyl ether
DMSO dimethyl sulphoxide
HATU 1-[Bis(dimethylamino)methylene]-1H-1,2,3-triazolo[4,5-b]pyridinium 3-oxid hexafluorophosphate
t_{R} retention time

### Example 1—Synthesis of N-[(2-fluorophenyl)methyl]-1-[(E)-phenylethenesulfonyl]azetidine-3-carboxamide

Synthetic Route 1

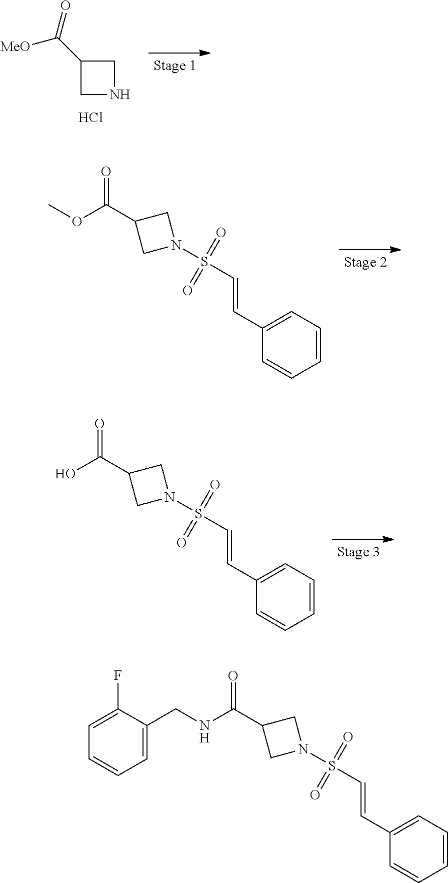 [SMILES: COC(=O)C1CNC1 ; Cl ; COC(=O)C1CN(S(=O)(=O)/C=C/c2ccccc2)C1 ; O=C(O)C1CN(S(=O)(=O)/C=C/c2ccccc2)C1 ; O=C(NCc1ccccc1F)C1CN(S(=O)(=O)/C=C/c2ccccc2)C1]

### Stage 1—Example 1—Methyl 1-[(E)-2-phenylethenesulfonyl]azetidine-3-carboxylate

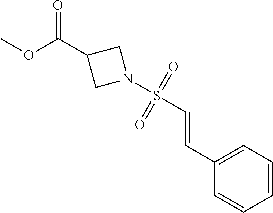 [SMILES: COC(=O)C1CN(S(=O)(=O)/C=C/c2ccccc2)C1]

To a solution of (E)-2-phenylethene-1-sulfonyl chloride (668.4 mg, 3.3 mmol) and DIPEA (1.09 mL, 6.6 mmol) in DCM (10 mL) was added portion wise methyl azetidine-3-carboxylate hydrochloride (500 mg, 3.3 mmol). The reaction was stirred at r.t. for 1 h. The solvent was removed under pressure and the resulting crude extract was purified by automated flash column chromatography (100% heptane to 50:50 heptane:EtOAc) to afford the required product as a white solid. Yield: 548 mg (59%)

LC/MS t_{R} 1.23 min; MS (ES⁺) m/z 282 [M+H]⁺ (D)

¹H NMR (500 MHz, CDCl₃) δ 7.61-7.52 (m, 3H), 7.46 (qd, 3H), 6.83 (d, 1H), 4.16-4.11 (m, 4H), 3.72 (s, 3H), 3.48-3.37 (m, 1H)

### Stage 2—1-[(E)-2-phenylethenesulfonyl]azetidine-3-carboxylic Acid

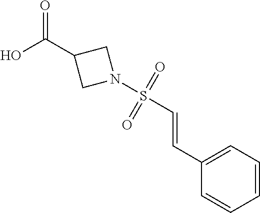 [SMILES: O=C(O)C1CN(S(=O)(=O)/C=C/c2ccccc2)C1]

To a solution of methyl 1-[(E)-2-phenylethenesulfonyl]azetidine-3-carboxylate (0.55 g, 1.95 mmol) in THF (16 mL) and water (4 mL) was added lithium hydroxide (139.9 mg, 5.84 mmol). The resulting suspension was stirred at r.t. for 15 h. The solvent was removed under reduced pressure and the aqueous extract acidified with AcOH (˜1 mL, pH<4). The aqueous extract was washed with EtOAc (3×15 mL). The organic extracts were combined, dried over anhydrous Na₂SO₄ and the solvent was removed under reduced pressure. The solid was azeotroped with heptanes (2×200 mL) to give the carboxylic acid as a white solid. Yield: 0.47 g (90%)

LC/MS t_{R} 1.12 min; MS (ES⁺) m/z 268 [M+H]⁺ (D)

¹H NMR (500 MHz, Methanol-d₄) δ 7.61-7.57 (m, 2H), 7.44 (d, 1H), 7.38-7.32 (m, 3H), 7.09 (d, 1H), 4.02-3.95 (m, 4H), 3.33 (tt, 1H)

### Stage 3—N-[(2-fluorophenyl)methyl]-1-[(E)-2-phenylethenesulfonyl]azetidine-3-carboxamide

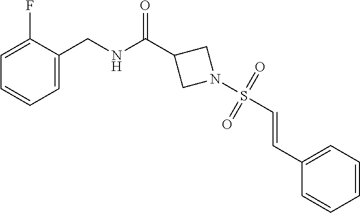 [SMILES: O=C(NCc1ccccc1F)C1CN(S(=O)(=O)/C=C/c2ccccc2)C1]

To a solution of 1-[(E)-2-phenylethenesulfonyl]azetidine-3-carboxylic acid (100 mg, 0.37 mmol), (2-fluorophenyl)methanamine (46.8 mg, 0.37 mmol) and DIPEA (0.25 mL, 1.5 mmol) in DMF (2 mL) was added HATU (284.5 mg, 0.75 mmol) and the solution stirred at r.t. for 15 h. The solvent was removed under reduced pressure and the crude product dissolved in (MeCN:DMSO, 2:1, 2 mL). Precipitation was promoted by dropwise addition of water. The precipitate was purified by column chromatography (DCM:MeOH, 197:3) to afford the required carboxamide as an off-white solid. Yield: 73.5 mg (52.5%)

LC/MS t_{R} 4.49 min; MS (ES⁺) m/z 418 [M+H]⁺ (A)

¹H NMR (500 MHz, DMSO-d₆) δ 8.47 (t, 1H), 7.80 (dd, 2H), 7.52-7.45 (m, 5H), 7.34-7.23 (m, 2H), 7.17-7.08 (m, 2H), 4.28 (d, 2H), 3.97-3.92 (m, 4H), 3.44-3.37 (m, 1H)

### Example 2—Synthesis of N-(2-chlorophenyl)-1-[(E)-2-phenylethenesulfonyl]piperidine-4-carboxamide

Synthetic Route 2

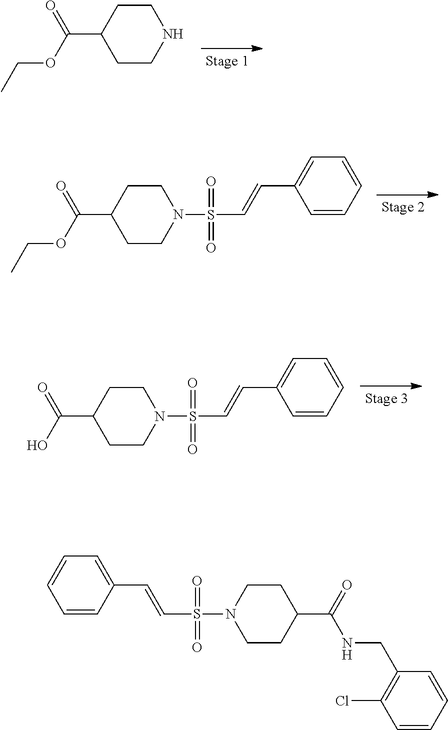 [SMILES: CCOC(=O)C1CCNCC1 ; CCOC(=O)C1CCN(S(=O)(=O)/C=C/c2ccccc2)CC1 ; O=C(O)C1CCN(S(=O)(=O)/C=C/c2ccccc2)CC1 ; O=C(NCc1ccccc1Cl)C1CCN(S(=O)(=O)/C=C/c2ccccc2)CC1]

### Stage 1—Ethyl 1-[(E)-2-phenylethenesulfonyl]piperidine-4-carboxylate

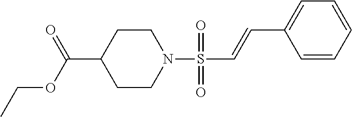 [SMILES: CCOC(=O)C1CCN(S(=O)(=O)/C=C/c2ccccc2)CC1]

To a solution of (E)-2-phenylethene-1-sulfonyl chloride (1.29 g, 6.36 mmol) in DCM (25 mL) at r.t. was added DIPEA (2.1 mL, 12.72 mmol). Ethyl piperidine-4-carboxylate (1 g, 6.36 mmol) was added dropwise to the stirred solution. The reaction was stirred at r.t. for 15 h. The solvent was removed under reduced pressure and the resulting crude product was purified by automated flash column chromatography (100% heptane to 50:50 heptane:EtOAc) to afford the requires ester as a white solid. 1.84 g (89%)

LC/MS t_{R} 1.40 min; MS (ES⁺) m/z 324 [M+H]⁺ (D)

¹H NMR (500 MHz, CDCl₃) δ 7.56-7.37 (m, 6H), 6.68 (d, 1H), 4.16 (q, 2H), 3.77-3.63 (m, 2H), 2.90-2.76 (m, 2H), 2.48-2.34 (m, 1H), 2.13-1.97 (m, 2H), 1.95-1.80 (m, 2H), 1.27 (t, 3H)

### Stage 2—1-[(E)-2-phenylethenesulfonyl]piperidine-4-carboxylic Acid

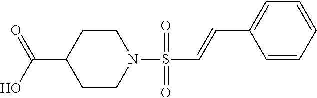 [SMILES: O=C(O)C1CCN(S(=O)(=O)/C=C/c2ccccc2)CC1]

To ethyl 1-[(E)-2-phenylethenesulfonyl]piperidine-4-carboxylate (1.8 g, 5.57 mmol) in THF (40 mL) and water (10 mL) was added lithium hydroxide (399.9 mg, 16.7 mmol). The reaction was stirred for 4 h. The reaction was acidified with AcOH (˜10 mL, pH<4). The solution was concentrated in-vacuo until only the aqueous remained. The aqueous layer was extracted with EtOAc (3×20 mL), The organic extracts were combined and dried over anhydrous Na₂SO₄. The solvent was removed in-vacuo, the white solid obtained was azeotroped with heptane (3×150 mL) to remove residual AcOH to afford the required acid as a white solid. Yield: 1.64 g (100%)

LC/MS t_{R} 1.21 min; MS (ES⁺) m/z 296 [M+H]⁺ (D)

¹H NMR (500 MHz, Methanol-d₄) δ 7.68-7.60 (m, 2H), 7.48-7.42 (m, 4H), 7.03 (d, 1H), 3.71-3.64 (m, 2H), 2.90-2.81 (m, 2H), 2.49-2.41 (m, 1H), 2.07-1.99 (m, 2H), 1.82-1.68 (m, 2H)

### Stage 3—N-(2-chlorophenyl)-1-[(E)-2-phenylethenesulfonyl]piperidine-4-carboxamide

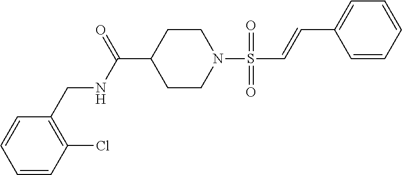 [SMILES: O=C(NCc1ccccc1Cl)C1CCN(S(=O)(=O)/C=C/c2ccccc2)CC1]

To a solution of 1-[(E)-2-phenylethenesulfonyl]piperidine-4-carboxylic acid (100 mg, 0.34 mmol), (2-chlorophenyl)methanamine (52.74 mg, 0.37 mmol) and DIPEA (0.22 mL, 1.35 mmol) in DMF (2 mL) was added HATU (257.5 mg, 0.68 mmol) and the reaction was stirred overnight at r.t. for 15 h. The solvent was removed in-vacuo and the resulting crude product purified by basic prep. chromatography to afford the required compound as a white solid. Yield: 45.4 mg (32%)

LC/MS t_{R} 4.49 min; MS (ES⁺) m/z 418 [M+H]⁺ (A)

¹H NMR (500 MHz, CDCl₃) δ 7.53-7.35 (m, 8H), 7.27-7.22 (m, 2H), 6.67 (d, 1H), 5.92 (s, 1H), 4.54 (d, 2H), 3.80 (dt, 2H), 2.78 (td, 2H), 2.23 (tt, 1H), 1.97 (dd, 2H), 1.92-1.81 (m, 2H)

### Example 3—Synthesis of 1-[(E)-2-phenylethenesulfonyl]-N-(pyridin-2-ylmethyl)piperidine-4-carboxamide

### Stage 1—1-[(E)-2-phenylethenesulfonyl]-N-(pyridin-2-ylmethyl)piperidine-4-carboxamide

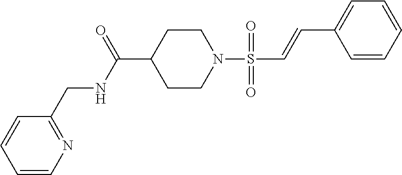 [SMILES: O=C(NCc1ccccn1)C1CCN(S(=O)(=O)/C=C/c2ccccc2)CC1]

To a solution of 1-[(E)-2-phenylethenesulfonyl]piperidine-4-carboxylic acid (100 mg, 0.34 mmol), pyridin-2-ylmethanamine (40.3 mg, 0.37 mmol) and DIPEA (0.22 mL, 1.35 mmol) in DMF (2 mL) was added HATU (257.5 mg, 0.68 mmol) and the solution stirred at r.t. for 15 h. The solvent was removed in-vacuo and the crude extract was purified by basic prep. chromatography to afford the required product as a white solid. Yield: 96.4 mg (74%)

LC/MS t_{R} 3.69 min; MS (ES⁺) m/z 386 [M+H]⁺ (A)

¹H NMR (500 MHz, CDCl₃) δ 8.54 (d, 1H), 7.68 (td, 1H), 7.55-7.39 (m, 6H), 7.28-7.19 (m, 2H), 6.95 (s, 1H), 6.69 (d, 1H), 4.56 (d, 2H), 3.83 (dt, 2H), 2.81 (td, 2H), 2.34 (tt, 1H), 2.03 (dd, 2H), 1.99-1.85 (m, 2H)

### Example 4—Synthesis of N-[(2-fluorophenyl)methyl]-1-(4-methoxy benzenesulfonyl) piperidine-4-carboxamide

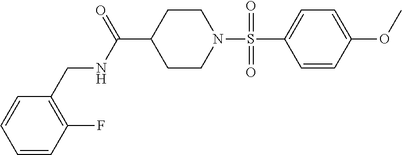 [SMILES: COc1ccc(S(=O)(=O)N2CCC(C(=O)NCc3ccccc3F)CC2)cc1]

To a solution of 4-methoxybenzene-1-sulfonyl chloride (83 mg, 0.40 mmol) in DCM (2 ml) was added N,N-diisopropylethylamine (0.30 ml, 1.83 mmol) and N-[(2-fluorophenyl)methyl]piperidine-4-carboxamide (hydrochloride salt, 100 mg, 0.37 mmol). The reaction was stirred at r.t. for 2.5 h. The reaction mixture was washed with saturated sodium bicarbonate (2 ml). The organic layer was dried over anhydrous MgSO₄, filtered and solvent removed in-vacuo. The crude product was purified by silica flash column chromatography (100% TBME) to give the title compound as colourless oil. Yield: 48 mg (32%)

LC/MS t_{R} 3.89 min; MS (ES⁺) m/z 407 [M+H]⁺ (C)

¹H NMR (500 MHz, DMSO-d₆) δ 8.23 (t, 1H), 7.74-7.61 (m, 2H), 7.35-7.19 (m, 2H), 7.18-7.07 (m, 4H), 4.25 (d, 2H), 3.85 (s, 3H), 3.65-3.46 (m, 2H), 2.26 (td, 2H), 2.15 (tt, 1H), 1.77 (dd, 2H), 1.58 (qd, 2H)

### Example 5—N-(2-fluorophenyl)-1-[(E)-2-phenylethenesulfonyl]piperidine-4-carboxamide—Purchased from Enamine

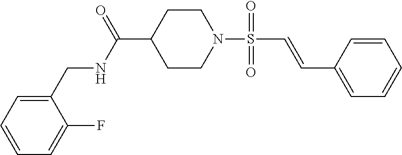 [SMILES: O=C(NCc1ccccc1F)C1CCN(S(=O)(=O)/C=C/c2ccccc2)CC1]

LC/MS t_{R} 3.25 min; MS (ES⁺) m/z 403 [M+H]⁺ (B)

¹H NMR (250 MHz, Methanol-d₄) δ 7.67-7.58 (i, 2H), 7.49-7.39 (i, 4H), 7.35-7.21 (m, 2H), 7.16-6.94 (i, 3H), 4.40 (s, 2H), 3.76 (dt, 2H), 2.74 (td, 2H), 2.44-2.23 (i, 1H), 1.96-1.66 (m, 4H)

### Example 6—N-[(2-fluorophenyl)methyl]-1-(benzenesulfonyl) piperidine-4-carboxamide—Purchased from Enamine

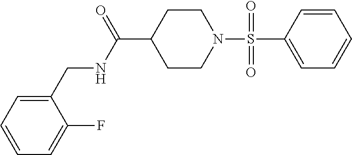 [SMILES: O=C(NCc1ccccc1F)C1CCN(S(=O)(=O)c2ccccc2)CC1]

LC/MS t_{R} 2.97 min; MS (ES⁺) m/z 377 [M+H]⁺ (B)

¹H NMR (250 MHz, Methanol-d₄) δ 7.83-7.72 (m, 2H), 7.74-7.53 (m, 3H), 7.35-7.19 (m, 2H), 7.18-6.97 (m, 2H), 4.37 (s, 2H), 3.77 (dt, 2H), 2.35 (td, 2H), 2.17 (dq, 1H), 1.93-1.64 (m, 4H)

### Example 7—N-[(2-fluorophenyl)methyl]-4-[(E)-2-phenylethenesulfonyl]piperazine-1-carboxamide

Synthetic Route 3

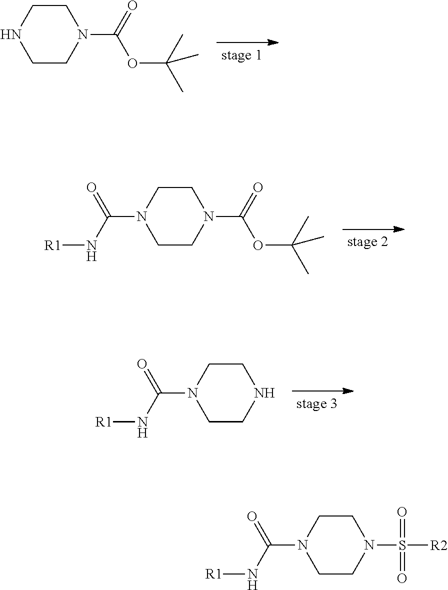

### Stage 1—tert-butyl 4-{[(2-fluorophenyl)methyl]carbamoyl}piperazine-1-carboxylate

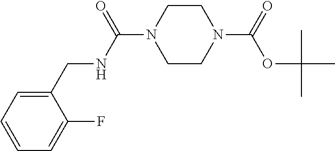 [SMILES: CC(C)(C)OC(=O)N1CCN(C(=O)NCc2ccccc2F)CC1]

A mixture of tert-butyl piperazine-1-carboxylate (100 mg, 0.54 mmol) and 1-fluoro-2-(isocyanatomethyl)benzene (97.4 mg, 0.64 mmol) in DCM (5 mL) was stirred at r.t. for 1.5 h. The reaction mixture was washed with saturated sodium bicarbonate (2 mL). The organic layer was dried over anhydrous MgSO₄, filtered and solvent removed in-vacuo. The crude product was purified by silica flash column chromatography (0-70% EtOAc:heptane) to give the title compound. Yield: 190 mg (100%)

LC/MS t_{R} 1.24 min; MS (ES⁺) m/z 360 [M+H]⁺ (D)

### Stage 2—N-[(2-fluorophenyl)methyl]piperazine-1-carboxamide

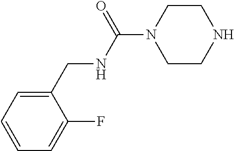 [SMILES: O=C(NCc1ccccc1F)N1CCNCC1]

To a solution of tert-butyl 4-{[(2-fluorophenyl)methyl]carbamoyl}piperazine-1-carboxylate (380 mg, 1.13 mmol) was added trifluoroacetic acid (0.86 ml, 11 mmol). The reaction was stirred at r.t. for 16 h. The reaction was quenched with saturated sodium carbonate. The organic layer was separated; the basic aqueous layer (pH 8) was washed with DCM (2×3 mL). The organic layers were combined, dried over anhydrous MgSO₄, filtered and solvent removed in-vacuo to give the title compound. Yield: 128 mg (48%)

LC/MS t_{R} 1.06 min; MS (ES⁺) m/z 238 [M+H]⁺ (A)

### Stage 3—N-[(2-fluorophenyl)methyl]-4-[(E)-2-phenylethenesulfonyl]piperazine-1-carboxamide

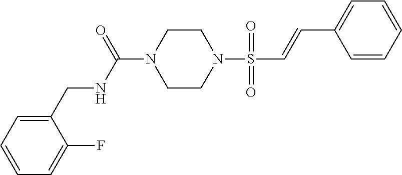 [SMILES: O=C(NCc1ccccc1F)N1CCN(S(=O)(=O)/C=C/c2ccccc2)CC1]

To a solution of (E)-2-phenylethene-1-sulfonyl chloride (56.37 mg, 0.28 mmol) in DCM (2 ml) was added N,N-diisopropylethylamine (0.17 ml, 1.01 mmol) followed by N-[(2-fluorophenyl)methyl]piperazine-1-carboxamide (60 mg, 0.25 mmol). The reaction was stirred at r.t. for 2 h. The reaction mixture was washed with saturated sodium bicarbonate (2 mL). The organic layer was dried over anhydrous MgSO₄, filtered and solvent removed in-vacuo. The crude product was purified by preparative HPLC (acidic) to give the title compound as white solid. Yield: 56.3 mg (54%)

LC/MS t_{R} 3.19 min; MS (ES⁺) m/z 404 [M+H]⁺ (B)

¹H NMR (500 MHz, DMSO-d₆) δ 7.76 (dd, 2H), 7.49-7.41 (m, 3H), 7.40 (s, 1H), 7.31 (s, 1H), 7.29-7.19 (m, 2H), 7.15 (t, 1H), 7.12-7.03 (m, 2H), 4.25 (d, 2H), 3.54-3.39 (m, 4H), 3.15-2.96 (m, 4H)

### Example 8—Synthesis of N-[(2-fluorophenyl)methyl]-N-methyl-1-[(E)-2-phenylethene sulfonyl]piperidine-4-carboxamide

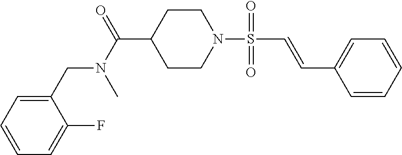 [SMILES: CN(Cc1ccccc1F)C(=O)C1CCN(S(=O)(=O)/C=C/c2ccccc2)CC1]

1-[(E)-2-phenylethenesulfonyl]piperidine-4-carboxylic acid (100 mg, 0.34 mmol), DCM (3 ml), [(2-fluorophenyl)methyl](methyl)amine (0.34 ml, 0.68 mmol) and N,N-diisopropylethylamine (0.09 ml, 0.51 mmol) were added to a flask. The mixture was stirred for 5 min. (1-[Bis(dimethylamino)methylene]-1H-1,2,3-triazolo[4,5-b]pyridinium 3-oxid hexafluorophosphate (HATU, 141.61 mg, 0.37 mmol) was added and the reaction stirred at r.t. for 3 h. The reaction mixture was washed with saturated sodium bicarbonate (2 mL). The organic layer was dried over anhydrous MgSO₄, filtered and solvent removed in-vacuo. The crude product was purified by silica flash column chromatography (40-100% TBME/heptane) to give the title compound as yellow oil. Yield: 113.6 mg (80%)

LC/MS t_{R} 4.28 min; MS (ES⁺) m/z 417 [M+H]⁺ (C)

¹H NMR (250 MHz, DMSO-d₆) δ 7.90-7.62 (m, 2H), 7.60-6.92 (m, 9H), 4.60 (d, 2H), 3.59 (t, 2H), 2.98 (s, 2H), 2.92-2.59 (m, 4H), 1.94-1.44 (m, 4H)

### Example 9—Synthesis of 1-(2H-chromene-3-sulfonyl)-N-[(2-fluorophenyl)methyl]piperidine-4-carboxamide

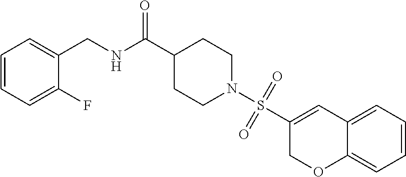 [SMILES: O=C(NCc1ccccc1F)C1CCN(S(=O)(=O)C2=Cc3ccccc3OC2)CC1]

To a solution of 2H-chromene-3-sulfonyl chloride (93.03 mg, 0.40 mmol) in DCM (2 ml) was added N,N-diisopropylethylamine (0.30 ml, 1.83 mmol)) followed by N-[(2-fluorophenyl)methyl]piperidine-4-carboxamide (hydrochloride salt) (100 mg, 0.37 mmol). The reaction was stirred at r.t. for 16 h. The reaction mixture was washed with saturated sodium bicarbonate (2 mL). The organic layer was dried over anhydrous MgSO₄, filtered and the solvent removed in-vacuo. The crude product was purified by silica flash column chromatography (35-100% EtOAc:heptane) to give the title compound. Yield: 109.8 mg (70%)

LC/MS t_{R} 3.86 min; MS (ES⁺) m/z 431 [M+H]⁺ (B)

¹H NMR (500 MHz, DMSO-d₆) δ 8.33 (t, 1H), 7.42 (dd, 1H), 7.38-7.22 (m, 4H), 7.19-7.10 (m, 2H), 7.02 (t, 1H), 6.92 (d, 1H), 4.89 (s, 2H), 4.28 (d, 2H), 3.61 (d, 2H), 2.76 (td, 2H), 2.33 (ddt, 1H), 1.90-1.76 (m, 2H), 1.60 (m, 2H)

### Example 10—Synthesis of [(2-fluorophenyl)methyl]({1-[(E)-phenylethenesulfonyl]piperidin-4-yl}methyl)amine I

Synthetic Route 4

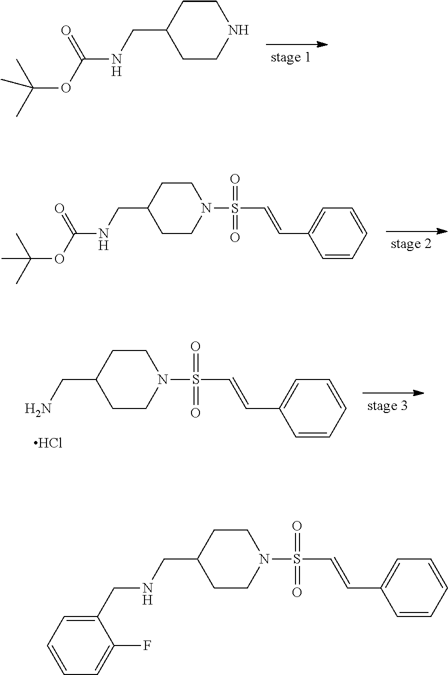 [SMILES: CC(C)(C)OC(=O)NCC1CCNCC1 ; CC(C)(C)OC(=O)NCC1CCN(S(=O)(=O)/C=C/c2ccccc2)CC1 ; NCC1CCN(S(=O)(=O)/C=C/c2ccccc2)CC1 ; Cl ; O=S(=O)(/C=C/c1ccccc1)N1CCC(CNCc2ccccc2F)CC1]

### Stage 1—tert-butyl N-({1-[(E)-2-phenylethenesulfonyl]piperidin-4-yl}methyl)carbamate

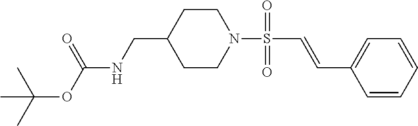 [SMILES: CC(C)(C)OC(=O)NCC1CCN(S(=O)(=O)/C=C/c2ccccc2)CC1]

To a solution of (E)-2-phenylethene-1-sulfonyl chloride (104 mg, 0.51 mmol) in DCM (5 ml) was added N,N-diisopropylethylamine (0.39 ml, 2.33 mmol) followed by tert-butyl N-(piperidin-4-ylmethyl)carbamate (100 mg, 0.47 mmol). The reaction was stirred at r.t. for 2.5 h. The reaction mixture was washed with saturated sodium bicarbonate (2 mL). The organic layer was dried over anhydrous MgSO₄, filtered and solvent removed in-vacuo. The crude product was purified by silica flash column chromatography (0-50% EtOAc:heptane) to give the title compound as white solid. Yield: 175 mg (99%)

LC/MS t_{R} 1.38 min; MS (ES⁺) m/z 403 [M+H]⁺ (D)

### Stage 2—N-({1-[(E)-2-phenylethenesulfonyl]piperidin-4-yl}methyl)carbamate (Hydrochloride Salt)

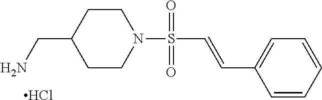 [SMILES: NCC1CCN(S(=O)(=O)/C=C/c2ccccc2)CC1 ; Cl]

To a solution of tert-butyl N-({1-[(E)-2-phenylethenesulfonyl]piperidin-4-yl}methyl)carbamate (175 mg, 0.46 mmol) was added 4N HCl in dioxane (1.15 ml, 5.0 mmol). The reaction was stirred at r.t. for 16 h. The reaction mixture was evaporated to dryness to give the title compound. The material was used in the next step without further purification. Yield: 158 mg, (108%)

LC/MS t_{R} 1.39 min; MS (ES⁺) m/z 281 [M+H]⁺ (A)

### Stage 3—[(2-fluorophenyl)methyl]({1-[(E)-2-phenylethenesulfonyl]piperidin-4-yl}methyl)amine

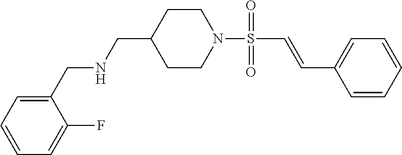 [SMILES: O=S(=O)(/C=C/c1ccccc1)N1CCC(CNCc2ccccc2F)CC1]

To a solution of {1-[(E)-2-phenylethenesulfonyl]piperidin-4-yl}methanamine (hydrochloride salt) (92 mg, 0.33 mmol) in DCM (2 ml) was added 2-fluorobenzaldehyde (0.04 ml, 0.39 mmol) followed by acetic acid (0.02 ml, 0.39 mmol). The reaction was stirred at r.t. for 2 h. Sodium triacetoxyborohydride (277 mg, 1.31 mmol) was added, followed by acetic acid (0.07 ml, 1.32 mmol). The reaction was stirred at r.t. for 16 h. The reaction was quenched with saturated sodium carbonate. The organic layer was separated, dried over anhydrous MgSO₄, filtered and solvent removed in-vacuo. The crude product was purified by silica flash column chromatography (0-100%, EtOAc:heptane) to give the title compound as beige solid. Yield: 64.3 mg (32%)

LC/MS t_{R} 2.05 min; MS (ES⁺) m/z 389 [M+H]⁺ (B)

¹H NMR (500 MHz, DMSO-d₆) δ 7.82-7.69 (m, 2H), 7.44 (dt, 4H), 7.37 (d, 1H), 7.31-7.22 (m, 2H), 7.18-7.06 (m, 2H), 3.70 (s, 2H), 3.56 (d, 2H), 2.63 (td, 2H), 2.38 (d, 2H), 1.80 (d, 2H), 1.61-1.41 (m, 1H), 1.27-1.08 (m, 2H)

The following compounds were further manufactured in order to investigate their functional activity. Table 4 summarizes the names of the manufactured compounds.

TABLE 4
Compound names

| Example | Compound name |
|---|---|
| 11 | 1-[(E)-2-phenylethenesulfonyl]-N-{[2- |
| | (trifluoromethyl)phenyl]methyl}piperidine-4-carboxamide |
| 12 | 1-(4-tert-butylbenzenesulfonyl)-N-[(2- |
| | fluorophenyl)methyl]piperidine-4-carboxamide |
| 13 | 1-(4-cyanobenzenesulfonyl)-N-[(2- |
| | fluorophenyl)methyl]piperidine-4-carboxamide |
| 14 | N-[(1H-1,3-benzodiazol-5-yl)methyl]-1-[(E)-2- |
| | phenylethenesulfonyl]piperidine-4-carboxamide |
| 15 | N-[(2-methoxyphenyl)methyl]-1-[(E)-2- |
| | phenylethenesulfonyl]piperidine-4-carboxamide |
| 16 | N-[(2-fluorophenyl)methyl]-1-(3- |
| | methoxybenzenesulfonyl)piperidine-4-carboxamide |
| 17 | N-[(2-fluorophenyl)methyl]-1-(4- |
| | nitrobenzenesulfonyl)piperidine-4-carboxamide |
| 18 | N-[(2-methanesulfonylphenyl)methyl]-1-[(E)-2- |
| | phenylethenesulfonyl]piperidine-4-carboxamide |
| 19 | N-[(2-fluorophenyl)methyl]-1-(2- |
| | methoxybenzenesulfonyl)piperidine-4-carboxamide |
| 20 | 1-(4-acetamidobenzenesulfonyl)-N-[(2- |
| | fluorophenyl)methyl]piperidine-4-carboxamide |
| 21 | N-[(3-fluorophenyl)methyl]-1-[(E)-2- |
| | phenylethenesulfonyl]piperidine-4-carboxamide |
| 22 | N-[(2,3-difluorophenyl)methyl]-1-[(E)-2- |
| | phenylethenesulfonyl]piperidine-4-carboxamide |
| 23 | N-[(2,5-difluorophenyl)methyl]-1-[(E)-2- |
| | phenylethenesulfonyl]piperidine-4-carboxamide |
| 24 | N-[(2-fluorophenyl)methyl]-1-[4-(propan-2- |
| | yloxy)benzenesulfonyl]piperidine-4-carboxamide |
| 25 | N-[(2-fluorophenyl)methyl]-1-[4- |
| | (trifluoromethyl)benzenesulfonyl]piperidine-4-carboxamide |
| 26 | 1-(2H-1,3-benzodioxole-5-sulfonyl)-N-[(2- |
| | fluorophenyl)methyl]piperidine-4-carboxamide |
| 27 | 1-(4-chlorobenzenesulfonyl)-N-[(2- |
| | fluorophenyl)methyl]piperidine-4-carboxamide |
| 28 | 1-(3,4-dichlorobenzenesulfonyl)-N-[(2- |
| | fluorophenyl)methyl]piperidine-4-carboxamide |
| 29 | N-[(2-fluorophenyl)methyl]-1-[4- |
| | (trifluoromethoxy)benzenesulfonyl]piperidine-4-carboxamide |
| 30 | 1-{[1,1′-biphenyl]-4-sulfonyl}-N-[(2- |
| | fluorophenyl)methyl]piperidine-4-carboxamide |
| 31 | N-[(2-methylphenyl)methyl]-1-[(E)-2- |
| | phenylethenesulfonyl]piperidine-4-carboxamide |
| 32 | N-[(2-bromophenyl)methyl]-1-[(E)-2- |
| | phenylethenesulfonyl]piperidine-4-carboxamide |
| 33 | 1-(3-tert-butylbenzenesulfonyl)-N-[(2- |
| | fluorophenyl)methyl]piperidine-4-carboxamide |
| 34 | 1-(4-ethylbenzenesulfonyl)-N-[(2- |
| | fluorophenyl)methyl]piperidine-4-carboxamide |
| 35 | N-[(2-fluorophenyl) methyl]-1 -(4- |
| | methylbenzenesulfonyl)piperidine-4-carboxamide |
| 36 | N-[(2-fluorophenyl)methyl]-1-(4- |
| | iodobenzenesulfonyl)piperidine-4-carboxamide |
| 37 | 1-(4-tert-butylbenzenesulfonyl)-N-[(pyridin-2- |
| | yl)methyl]piperidine-4-carboxamide |
| 38 | 1-(4-tert-butylbenzenesulfonyl)-N-[(2- |
| | methoxyphenyl)methyl]piperidine-4-carboxamide |
| 39 | 1-(4-tert-butylbenzenesulfonyl)-N-[(2- |
| | chlorophenyl)methyl]piperidine-4-carboxamide |
| 40 | N-{[2-(morpholin-4-yl)phenyl]methyl}-1-[(E)-2- |
| | phenylethenesulfonyl]piperidine-4-carboxamide |
| 41 | N-[(2-hydroxyphenyl)methyl]-1-[(E)-2- |
| | phenylethenesulfonyl]piperidine-4-carboxamide |
| 42 | 1-(2-tert-butylbenzenesulfonyl)-N-[(2- |
| | fluorophenyl)methyl]piperidine-4-carboxamide |
| 43 | 1-(2,4-dichlorobenzenesulfonyl)-N-[(2- |
| | fluorophenyl)methyl]piperidine-4-carboxamide |
| 44 | 1-(3,5-dichlorobenzenesulfonyl)-N-[(2- |
| | fluorophenyl)methyl]piperidine-4-carboxamide |
| 45 | 1-(4-fluorobenzenesulfonyl)-N-[(2- |
| | fluorophenyl)methyl]piperidine-4-carboxamide |
| 46 | 1-(4-tert-butylbenzenesulfonyl)-N-[(2- |
| | fluorophenyl)methyl]azetidine-3-carboxamide |
| 47 | 1-[(2,2-difluoro-2H-1,3-benzodioxol-5-yl)sulfonyl]-N-[(2- |
| | fluorophenyl)methyl]piperidine-4-carboxamide |
| 48 | N-[(2-fluoro-4-methoxyphenyl)methyl]-1-[(E)-2- |
| | phenylethenesulfonyl]piperidine-4-carboxamide |
| 49 | 1-(4-tert-butylbenzenesulfonyl)-N-[(2- |
| | chlorophenyl)methyl]azetidine-3-carboxamide |
| 50 | 1-(4-tert-butylbenzenesulfonyl)-N-[(2- |
| | methoxyphenyl)methyl]azetidine-3-carboxamide |
| 51 | 1-(4-tert-butylbenzenesulfonyl)-N-[(pyridin-2- |
| | yl)methyl]azetidine-3-carboxamide |
| 52 | N-[(2-fluorophenyl)methyl]-1-[4- |
| | (trifluoromethyl)benzenesulfonyl]azetidine-3-carboxamide |
| 53 | N-[(2-cyanophenyl)methyl]-1-[(E)-2- |
| | phenylethenesulfonyl]piperidine-4-carboxamide |
| 54 | 1-[(E)-2-phenylethenesulfonyl]-N-{[2-(propan-2- |
| | yloxy)phenyl]methyl}piperidine-4-carboxamide |
| 55 | 1-(3,5-dichlorobenzenesulfonyl)-N-[(pyridin-2- |
| | yl)methyl]piperidine-4-carboxamide |
| 56 | N-[(4-fluorophenyl)methyl]-1-[(E)-2- |
| | phenylethenesulfonyl]piperidine-4-carboxamide |
| 57 | N-[(pyridin-2-yl)methyl]-1-[4- |
| | (trifluoromethyl)benzenesulfonyl]piperidine-4-carboxamide |
| 58 | N-[(pyridin-2-yl)methyl]-1-[4- |
| | (trifluoromethoxy)benzenesulfonyl]piperidine-4-carboxamide |
| 59 | 1-(3-chlorobenzenesulfonyl)-N-[(2- |
| | fluorophenyl)methyl]piperidine-4-carboxamide |
| 60 | 1-(3,5-dimethylbenzenesulfonyl)-N-[(pyridin-2- |
| | yl)methyl]piperidine-4-carboxamide |
| 61 | 1-(2,4-dimethoxybenzenesulfonyl)-N-[(pyridin-2- |
| | yl)methyl]piperidine-4-carboxamide |
| 62 | 1-(2,5-dichlorobenzenesulfonyl)-N-[(pyridin-2- |
| | yl)methyl]piperidine-4-carboxamide |
| 63 | 1-(2,6-dichlorobenzenesulfonyl)-N-[(pyridin-2- |
| | yl)methyl]piperidine-4-carboxamide |
| 64 | 1-[4-(dimethylamino)benzenesulfonyl]-N-[(pyridin-2- |
| | yl)methyl]piperidine-4-carboxamide |
| 65 | 1-[4-(dimethylamino)benzenesulfonyl]-N-[(2- |
| | fluorophenyl)methyl]piperidine-4-carboxamide |
| 66 | methyl 2-[({1-[(E)-2-phenylethenesulfonyl]piperidin-4- |
| | yl}formamido)methyl]benzoate |
| 67 | 1-(3,4-dichlorobenzenesulfonyl)-N-[(pyridin-2- |
| | yl)methyl]piperidine-4-carboxamide |
| 68 | 1-{[1,1′-biphenyl]-4-sulfonyl}-N-[(pyridin-2- |
| | yl)methyl]pipendine-4-carboxamide |
| 69 | N-{[2-(hydroxymethyl)phenyl]methyl}-1-[(E)-2- |
| | phenylethenesulfonyl]pipendine-4-carboxamide |
| 70 | N-[(pyridin-2-yl)methyl]-1-[4-(pyrrolidin-1- |
| | yl)benzenesulfonyl]pipendine-4-carboxamide |
| 71 | 1-(2-chlorobenzenesulfonyl)-N-[(pyridin-2- |
| | yl)methyl]pipendine-4-carboxamide |
| 72 | N-[(2-fluorophenyl)methyl]-1-(4- |
| | hydroxybenzenesulfonyl)pipendine-4-carboxamide |
| 73 | 1-(2,4-dichlorobenzenesulfonyl)-N-[(pyridin-2- |
| | yl)methyl]pipendine-4-carboxamide |
| 74 | 1-[2-chloro-4-(trifluoromethyl)benzenesulfonyl]-N-[(pyridin- |
| | 2-yl)methyl]piperidine-4-carboxamide |
| 75 | N-[(pyridin-2-yl)methyl]-1-[3- |
| | (trifluoromethyl)benzenesulfonyl]pipendine-4-carboxamide |
| 76 | 1-(3-nitrobenzenesulfonyl)-N-[(pyridin-2- |
| | yl) methyl]pipendine-4-carboxamide |
| 77 | 1-(4-carbamoylbenzenesulfonyl)-N-[(2- |
| | fluorophenyl)methyl]pipendine-4-carboxamide |
| 78 | 1-(3-chloro-5-fluorobenzenesulfonyl)-N-[(pyridin-2- |
| | yl)methyl]pipendine-4-carboxamide |
| 79 | 1-(3,5-dichlorobenzenesulfonyl)-N-[(2- |
| | hydroxyphenyl)methyl]pipendine-4-carboxamide |
| 80 | N-[(2-carbamoylphenyl)methyl]-1-(3,5- |
| | dichlorobenzenesulfonyl)pipendine-4-carboxamide |
| 81 | N-[(2-aminophenyl)methyl]-1-(3,5- |
| | dichlorobenzenesulfonyl)pipendine-4-carboxamide |
| 82 | N-[(2-hydroxyphenyl)methyl]-1-[4- |
| | (trifluoromethyl)benzenesulfonyl]pipendine-4-carboxamide |
| 83 | 1-(2,3-dichlorobenzenesulfonyl)-N-[(pyridin-2- |
| | yl)methyl]pipendine-4-carboxamide |
| 84 | 1-[3-fluoro-5-(trifluoromethyl)benzenesulfonyl]-N-[(pyridin-2- |
| | yl) methyl]pipendine-4-carboxamide |
| 85 | 1-[3-(pentafluoro-lambda6-sulfanyl)benzenesulfonyl]-N- |
| | [(pyridin-2-yl)methyl]pipendine-4-carboxamide |
| 86 | 1-[4-(pentafluoro-lambda6-sulfanyl)benzenesulfonyl]-N- |
| | [(pyridin-2-yl)methyl]pipendine-4-carboxamide |
| 87 | 1-(3,5-dichlorobenzenesulfonyl)-N-{[2- |
| | (hydroxymethyl)phenyl]methyl}piperidine-4-carboxamide |
| 88 | methyl 3-[(4-{[(2-fluorophenyl)methyl]carbamoyl}piperidin-1- |
| | yl)sulfonyl]benzoate |
| 89 | methyl 4-[(4-{[(2-fluorophenyl)methyl]carbamoyl}piperidin-1- |
| | yl)sulfonyl]benzoate |
| 90 | 3-[(4-{[(2-fluorophenyl)methyl]carbamoyl}piperidin-1- |
| | yl)sulfonyl]benzoic acid |
| 91 | 4-[(4-{[(2-fluorophenyl)methyl]carbamoyl}piperidin-1- |
| | yl)sulfonyl]benzoic acid |
| 92 | N-([2-(azidomethyl)phenyl]methyl)-1-(3,5- |
| | dichlorobenzenesulfonyl)piperidine-4-carboxamide |
| 93 | N-[(pyridin-2-yl)methyl]-1-[3- |
| | (trifluoromethoxy)benzenesulfonyl]piperidine-4-carboxamide |
| 94 | N-[(2-fluorophenyl)methyl]-1-(4- |
| | formylbenzenesulfonyl)piperidine-4-carboxamide |
| 95 | 1-(4-iodobenzenesulfonyl)-N-[(pyridin-2-yl)methyl]piperidine- |
| | 4-carboxamide |
| 96 | 1-(3-bromo-5-chlorobenzenesulfonyl)-N-[(pyridin-2- |
| | yl)methyl]piperidine-4-carboxamide |
| 97 | 1-(4-cyclopropylbenzenesulfonyl)-N-[(pyridin-2- |
| | yl)methyl]piperidine-4-carboxamide |
| 98 | N-([2-(aminomethyl)phenyl]methyl)-1-(3,5- |
| | dichlorobenzenesulfonyl)piperidine-4-carboxamide |
| 99 | N-[(pyridin-2-yl)methyl]-1-[4-(2,2,2- |
| | trifluoroethyl)benzenesulfonyl]piperidine-4-carboxamide |
| 100 | N-[(2-fluorophenyl)methyl]-1-[4- |
| | (hydroxymethyl)benzenesulfonyl]piperidine-4-carboxamide |
| 101 | 1-(3-chloro-5-cyclopropylbenzenesulfonyl)-N-[(pyridin-2- |
| | yl)methyl]piperidine-4-carboxamide |
| 102 | 1-(4-[2-(dimethylamino)ethoxy]benzenesulfonyl)-N-[(2- |
| | fluorophenyl)methyl]piperidine-4-carboxamide |
| 103 | N-benzyl-1-(3,5-dichlorobenzenesulfonyl)piperidine-4- |
| | carboxamide |
| 104 | N-([4-(hydroxymethyl)phenyl]methyl)-1-[4- |
| | (trifluoromethyl)benzenesulfonyl]piperidine-4-carboxamide |
| 105 | N-[(2-fluorophenyl)methyl]-1-{4-[(pyrrolidin-1- |
| | yl)methyl]benzenesulfonyl}piperidine-4-carboxamide |
| 106 | 1-(3-acetylbenzenesulfonyl)-N-[(2- |
| | fluorophenyl)methyl]piperidine-4-carboxamide |
| 107 | N-[(2-fluorophenyl)methyl]-1-[3-(1- |
| | hydroxyethyl)benzenesulfonyl]piperidine-4-carboxamide |
| 108 | N-[(2-fluorophenyl)methyl]-1-{3-[(1E)-1- |
| | (hydroxyimino)ethyl]benzenesulfonyl}piperidine-4- |
| | carboxamide |
| 109 | N-[(2-fluorophenyl)methyl]-1-{3-[1- |
| | (propylamino)ethyl]benzenesulfonyl}piperidine-4-carboxamide |
| 110 | N-[(2-fluorophenyl)methyl]-1-{3-[1- |
| | (methylamino)ethyl]benzenesulfonyl}piperidine-4-carboxamide |
| 111 | 1-[4-(propan-2-yl)benzenesulfonyl]-N-[(pyridin-2- |
| | yl)methyl]piperidine-4-carboxamide |
| 112 | 1-[3-(1-fluoroethyl)benzenesulfonyl]-N-[(2- |
| | fluorophenyl)methyl]piperidine-4-carboxamide |
| 113 | 1-{3-[1-(diethylamino)ethyl]benzenesulfonyl}-N-[(2- |
| | fluorophenyl)methyl]piperidine-4-carboxamide |
| 114 | 1-(3,5-dichlorobenzenesulfonyl)-N-[(3- |
| | hydroxyphenyl)methyl]piperidine-4-carboxamide |
| 115 | 1-(3,5-dichlorobenzenesulfonyl)-N-[(3- |
| | fluorophenyl)methyl]piperidine-4-carboxamide |
| 116 | 1-[3-chloro-5-(oxetan-3-yl)benzenesulfonyl]-N-[(pyridin-2- |
| | yl)methyl]piperidine-4-carboxamide |
| 117 | 1-(3,5-dichlorobenzenesulfonyl)-N-{[3- |
| | (hydroxymethyl)phenyl]methyl}piperidine-4-carboxamide |
| 118 | methyl 3-({[1-(3,5-dichlorobenzenesulfonyl)piperidin-4- |
| | yl]formamido}methyl)benzoate |
| 119 | 3-({[1-(3,5-dichlorobenzenesulfonyl)piperidin-4- |
| | yl]formamido}methyl)benzoic acid |
| 120 | 1-(3,5-dichlorobenzenesulfonyl)-N-[(pyridin-2- |
| | yl)methyl]azetidine-3-carboxamide |
| 121 | 1-(4-chloro-3-methylbenzenesulfonyl)-N-[(pyridin-2- |
| | yl)methyl]piperidine-4-carboxamide |
| 122 | 1-(3-chloro-4-methylbenzenesulfonyl)-N-[(pyridin-2- |
| | yl)methyl]piperidine-4-carboxamide |
| 123 | 1-(3,5-dichlorobenzenesulfonyl)-N-[(4- |
| | hydroxyphenyl)methyl]piperidine-4-carboxamide |
| 124 | N-[(4-aminophenyl)methyl]-1-(3,5- |
| | dichlorobenzenesulfonyl)piperidine-4-carboxamide |
| 125 | N-[(pyridin-2-yl)methyl]-1-{4-[1- |
| | (trifluoromethyl)cyclopropyl]benzenesulfonyl}piperidine-4- |
| | carboxamide |
| 126 | 1-[3-bromo-5-(trifluoromethyl)benzenesulfonyl]-N-[(pyridin-2- |
| | yl) methyl]piperidine-4-carboxamide |
| 127 | 1-[3,5-bis(trifluoromethyl)benzenesulfonyl]-N-[(pyridin-2- |
| | yl)methyl]piperidine-4-carboxamide |
| 128 | 1-(3,5-dichlorobenzenesulfonyl)-N-methyl-N-[(pyridin-2- |
| | yl)methyl]piperidine-4-carboxamide |
| 129 | 1-(4-cyclopentylbenzenesulfonyl)-N-[(pyridin-2- |
| | yl)methyl]piperidine-4-carboxamide |
| 130 | 1-[3-chloro-5-(trifluoromethyl)benzenesulfonyl]-N-[(pyridin-2- |
| | yl)methyl]piperidine-4-carboxamide |
| 131 | 1-(4-cyclohexylbenzenesulfonyl)-N-[(pyridin-2- |
| | yl)methyl]piperidine-4-carboxamide |
| 132 | 1-(3,5-dichlorobenzenesulfonyl)-N-[(4- |
| | fluorophenyl)methyl]piperidine-4-carboxamide |

Table 5 summarizes the results for functional activity of selected compounds as shown in table 1 for several compounds as listed in table 4:

TABLE 5
Results of assay for functional activity
of selected compounds shown in table 4.

| | Uptake of | | | | |
|---|---|---|---|---|---|
| | | | | | Citrate/ |
| | | | | | fatty acid |
| | Citrate | Citrate | Glutamate | Glucose | synthesis |
| | Transporter protein | | | | |
| | INDY | INDY | GLT-1 | GLUT1 | INDY |
| | Cell line | | | | |
| | HEK293 | HepG2 | HEK293 | Huh7 | HepG2 |
| Example | IC50 in μM | | | | |
| 11 | >20.0 | n.d. | n.d. | n.d. | n.d. |
| 12 | 0.2 | 1.0 | >20 | n.d. | 0.6 |
| 13 | 20.6 | n.d. | n.d. | n.d. | n.d. |
| 14 | >20.0 | n.d. | n.d. | n.d. | n.d. |
| 15 | 2.9 | 2.7 | n.d. | n.d. | n.d. |
| 16 | 1.4 | 1.5 | n.d. | n.d. | n.d. |
| 17 | >20.0 | n.d. | n.d. | n.d. | n.d. |
| 18 | >20.0 | n.d. | n.d. | n.d. | n.d. |
| 19 | 5.0 | 3.4 | n.d. | n.d. | n.d. |
| 20 | >20.0 | n.d. | n.d. | n.d. | n.d. |
| 21 | 7.4 | >20 | n.d. | n.d. | n.d. |
| 22 | 3.4 | 7.0 | n.d. | n.d. | n.d. |
| 23 | 4.6 | >20 | n.d. | n.d. | n.d. |
| 24 | 9.3 | 4.2 | n.d. | n.d. | n.d. |
| 25 | >20.0 | 0.3 | >20 | n.d. | 0.4 |
| 26 | 5.2 | 15.9 | n.d. | n.d. | n.d. |
| 27 | 3.0 | 2.4 | n.d. | n.d. | n.d. |
| 28 | >20.0 | 15.7 | n.d. | n.d. | n.d. |
| 29 | 0.5 | 0.4 | >20 | n.d. | n.d. |
| 30 | 0.8 | 0.8 | n.d. | n.d. | n.d. |
| 31 | 15.2 | n.d. | n.d. | n.d. | n.d. |
| 32 | 5.1 | 6.7 | n.d. | n.d. | n.d. |
| 33 | 0.5 | 1.0 | n.d. | n.d. | n.d. |
| 34 | 1.0 | 4.5 | n.d. | n.d. | n.d. |
| 35 | 1.5 | 2.9 | n.d. | n.d. | n.d. |
| 36 | 1.0 | 1.0 | n.d. | n.d. | n.d. |
| 37 | 0.8 | 1.1 | >20 | n.d. | n.d. |
| 38 | 0.3 | 0.8 | n.d. | n.d. | n.d. |
| 39 | 0.2 | 2.1 | n.d. | n.d. | n.d. |
| 40 | >20.0 | n.d. | n.d. | n.d. | n.d. |
| 41 | 1.8 | 3.2 | n.d. | n.d. | n.d. |
| 42 | >20.0 | n.d. | n.d. | n.d. | n.d. |
| 43 | 0.3 | 0.4 | n.d. | n.d. | n.d. |
| 44 | 0.1 | 0.1 | >20 | n.d. | 0.03 |
| 45 | 11.8 | n.d. | n.d. | n.d. | n.d. |
| 46 | 9.3 | 9.3 | n.d. | n.d. | n.d. |
| 47 | 0.7 | 0.7 | n.d. | n.d. | n.d. |
| 48 | >20.0 | n.d. | n.d. | n.d. | n.d. |
| 49 | 6.4 | 9.0 | >20 | n.d. | n.d. |
| 50 | 4.4 | 7.0 | >20 | n.d. | 6.2 |
| 51 | 14.4 | n.d. | n.d. | n.d. | n.d. |
| 52 | >20.0 | n.d. | n.d. | n.d. | n.d. |
| 53 | 15.9 | n.d. | n.d. | n.d. | n.d. |
| 54 | >20.0 | n.d. | n.d. | n.d. | n.d. |
| 55 | 0.8 | 0.4 | >20 | n.d. | 0.6 |
| 56 | >20.0 | n.d. | n.d. | n.d. | n.d. |
| 57 | 4.1 | 2.5 | >20 | n.d. | 2.5 |
| 58 | 6.4 | 2.7 | n.d. | n.d. | n.d. |
| 59 | 1.1 | 0.9 | n.d. | n.d. | n.d. |
| 60 | 1.3 | 0.7 | >20 | n.d. | n.d. |
| 61 | >20.0 | n.d. | n.d. | n.d. | n.d. |
| 62 | 8.2 | 4.6 | n.d. | n.d. | n.d. |
| 63 | >20.0 | n.d. | n.d. | n.d. | n.d. |
| 64 | >20.0 | n.d. | n.d. | n.d. | n.d. |
| 65 | >20.0 | n.d. | n.d. | n.d. | n.d. |
| 66 | 12.9 | n.d. | n.d. | n.d. | n.d. |
| 67 | 18.3 | 7.2 | n.d. | n.d. | n.d. |
| 68 | 19.7 | n.d. | n.d. | n.d. | n.d. |
| 69 | 11.7 | 4.5 | n.d. | n.d. | n.d. |
| 70 | >20.0 | n.d. | n.d. | n.d. | n.d. |
| 71 | 14.4 | n.d. | n.d. | n.d. | n.d. |
| 72 | >20.0 | n.d. | n.d. | n.d. | n.d. |
| 73 | 2.4 | 1.6 | >20 | n.d. | n.d. |
| 74 | 7.1 | 5.1 | n.d. | n.d. | n.d. |
| 75 | 5.7 | 3.3 | n.d. | n.d. | n.d. |
| 76 | >20.0 | n.d. | n.d. | n.d. | n.d. |
| 77 | >20.0 | n.d. | n.d. | n.d. | n.d. |
| 78 | 4.9 | 2.4 | n.d. | n.d. | n.d. |
| 79 | 0.2 | 0.1 | >20 | n.d. | n.d. |
| 80 | >20.0 | n.d. | n.d. | n.d. | n.d. |
| 81 | 0.4 | 0.1 | >20 | n.d. | n.d. |
| 82 | 1.1 | 0.6 | >20 | n.d. | n.d. |
| 83 | >20.0 | n.d. | n.d. | n.d. | n.d. |
| 84 | 4.3 | 2.2 | n.d. | n.d. | n.d. |
| 85 | 6.4 | 3.0 | n.d. | n.d. | n.d. |
| 86 | 1.1 | 0.4 | >20 | n.d. | 0.4 |
| 87 | 0.6 | 0.2 | >20 | n.d. | 0.6 |
| 88 | >20.0 | n.d. | n.d. | n.d. | n.d. |
| 89 | >20.0 | n.d. | n.d. | n.d. | n.d. |
| 90 | >20.0 | n.d. | n.d. | n.d. | n.d. |
| 91 | >20.0 | n.d. | n.d. | n.d. | n.d. |
| 92 | 1.0 | 0.8 | >20 | n.d. | n.d. |
| 93 | 16.4 | n.d. | n.d. | n.d. | n.d. |
| 94 | >20.0 | n.d. | n.d. | n.d. | n.d. |
| 95 | 5.5 | 2.4 | n.d. | n.d. | n.d. |
| 96 | 0.4 | 0.3 | >20 | n.d. | n.d. |
| 97 | 9.2 | 3.8 | n.d. | n.d. | n.d. |
| 98 | >20.0 | n.d. | n.d. | n.d. | n.d. |
| 99 | 6.3 | 3.2 | n.d. | n.d. | n.d. |
| 100 | >20.0 | n.d. | n.d. | n.d. | n.d. |
| 101 | 0.3 | 0.1 | >20 | n.d. | 0.2 |
| 102 | >20.0 | n.d. | n.d. | n.d. | n.d. |
| 103 | 0.1 | 0.1 | >20 | n.d. | n.d. |
| 104 | >20.0 | n.d. | n.d. | n.d. | n.d. |
| 105 | >20.0 | n.d. | n.d. | n.d. | n.d. |
| 106 | 11.1 | n.d. | n.d. | n.d. | n.d. |
| 107 | 13.2 | n.d. | n.d. | n.d. | n.d. |
| 108 | 20.4 | n.d. | n.d. | n.d. | n.d. |
| 109 | >20.0 | n.d. | n.d. | n.d. | n.d. |
| 110 | >20.0 | n.d. | n.d. | n.d. | n.d. |
| 111 | 2.7 | 5.2 | >20 | n.d. | n.d. |
| 112 | 1.4 | 0.9 | >20 | n.d. | n.d. |
| 113 | >20.0 | n.d. | n.d. | n.d. | n.d. |
| 114 | 0.7 | 0.6 | n.d. | n.d. | n.d. |
| 115 | 0.2 | 0.1 | n.d. | n.d. | n.d. |
| 116 | 5.9 | 2.3 | n.d. | n.d. | n.d. |
| 117 | 0.8 | 0.5 | n.d. | n.d. | n.d. |
| 118 | 5.7 | 4.0 | n.d. | n.d. | n.d. |
| 119 | >20.0 | n.d. | n.d. | n.d. | n.d. |
| 120 | 7.5 | 2.8 | n.d. | n.d. | n.d. |
| 121 | 18.8 | n.d. | n.d. | n.d. | n.d. |
| 122 | 18.8 | n.d. | n.d. | n.d. | n.d. |
| 123 | 2.0 | 1.3 | n.d. | n.d. | n.d. |
| 124 | 1.8 | 0.9 | n.d. | n.d. | n.d. |
| 125 | 0.8 | 0.5 | n.d. | n.d. | n.d. |
| 126 | 0.5 | 0.4 | n.d. | n.d. | n.d. |
| 127 | 3.2 | 1.2 | n.d. | n.d. | n.d. |
| 128 | >20.0 | n.d. | n.d. | n.d. | n.d. |
| 129 | 1.6 | 7.4 | n.d. | n.d. | n.d. |
| 130 | 0.7 | 0.5 | n.d. | n.d. | n.d. |
| 131 | 6.1 | 4.7 | n.d. | n.d. | n.d. |
| 132 | 2.0 | 0.7 | n.d. | n.d. | n.d. |

The compounds listed in table 4 and investigated as shown in table 5 are within the scope of the present invention and will be further investigated for their potential use in the treatment of diseases.

1. A compound for use in a method for treating diseases depending on the activity of a citrate transporter, wherein the compound has the general formula (I): 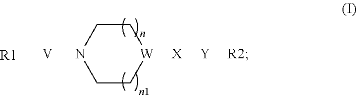
wherein
R1 is Aryl, substituted aryl, styryl, or bicyclic;
V is SO₂;
W is N with n and n1 are 1 and X is C═O, or
W is C with n and n1 are 0 or 1 and X is CH₂ or C═O;
Y is N—H or N-methyl; and
R2 is benzyl, substituted benzyl or CH₂(2-pyridyl);
with the proviso that the compound of formula (I) is not the following compound:

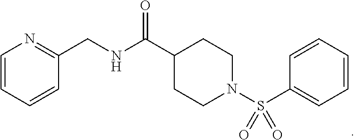 [SMILES: O=C(NCc1ccccn1)C1CCN(S(=O)(=O)c2ccccc2)CC1]

2. The compound of claim 1, wherein the compound is 1-(2-Phenyl-ethenesulfonyl)-azetidine-3-carboxylic acid 2-fluoro-benzylamide or 1-(3,5-dichlorobenzenesulfonyl)-N-[(4-fluorophenyl)methyl]piperidine-4-carboxamide.

3. The compound of claim 1, wherein the compound inhibits the activity of the citrate transporter directly or allosterically.

4. A compound, wherein the compound is 1-(2-Phenyl-ethenesulfonyl)-azetidine-3-carboxylic acid 2-fluoro-benzylamide.

5. A compound of claim 1 including pharmaceutically applicable salts, tautomers and stereoisomers of the compound, including mixtures thereof in all ratios for use in the treatment and/or prevention of diseases depending on the activity of a citrate transporter.

6. The compound of claim 5, wherein the citrate transporter is the gene product of Indy or a homologue thereof.

7. A compound according to claim 1 for the treatment and/or prevention of
a. metabolic diseases selected from the group comprising insulin resistance, alcoholic and non-alcoholic fatty liver disease, non-alcoholic steatohepatitis (NASH), obesity, type 1 diabetes, type 2 diabetes, dyslipidemia, hereditary diseases and metabolic syndrome, and
b. eating disorders, and
c. chronic liver diseases, and
d. liver cancer and cancer related to obesity, and
e. drug induced hepatic steatosis.

8. (canceled)

9. A method for the treatment and/or prevention of age-related diseases or atherosclerosis and cardiovascular disease, cancer, arthritis, cataracts, osteoporosis, type 2 diabetes, hypertension, drug induced hepatic steatosis and neurodegenerative diseases like Alzheimer's disease comprising the step of administering a compound of general formula (I): 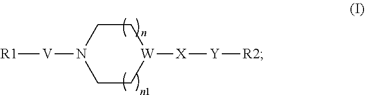
wherein
R1 is Aryl, substituted aryl, styryl, or bicyclic;
V is SO₂;
W is N with n and n1 are 1 and X is C═O, or
W is C with n and n1 are 0 or 1 and X is CH₂ or C═O;
Y is N—H or N-methyl; and
R2 is benzyl, substituted benzyl or CH₂(2-pyridyl).

10. (canceled)

11. (canceled)

12. (canceled)

13. (canceled)

14. (canceled)
